(19) United States
(12) Patent Application Publication
(10) Pub. No.: US 2017/0001872 A1
(43) Pub. Date: Jan. 5, 2017

(54) CIT-10: A TWO DIMENSIONAL LAYERED CRYSTALLINE MICROPOROUS SILICATE COMPOSITION AND COMPOSITIONS DERIVED THEREFROM

(71) Applicant: CALIFORNIA INSTITUTE OF TECHNOLOGY, Pasadena, CA (US)

(72) Inventors: JOEL E. SCHMIDT, UTRECHT (NL); MARK E. DAVIS, PASADENA, CA (US)

(21) Appl. No.: 15/198,219

(22) Filed: Jun. 30, 2016

Related U.S. Application Data
(60) Provisional application No. 62/187,074, filed on Jun. 30, 2015.

(51) Int. Cl. C01B 39/00; C10G 47/02; C07C 5/32; C07C 5/41; C07C 5/22; C07C 2/66; C07C 2/12; C10G 2/00; C01B 33/12; B01J 29/04; B01J 20/10; B01D 53/86; B01D 53/04; C07D 233/58; C10G 11/02

(52) U.S. Cl. CPC C01B 39/00; C10G 11/02; C10G 47/02; C07C 5/321; C07C 5/41; C07C 5/22; C07C 2/66; C07C 2/12; C10G 2/334; C01B 33/126; B01J 29/041; B01J 20/103; B01D 53/8628; B01D 53/04; C07D 233/58; C07C 2529/04; B01D 2255/50; B01D 2253/108

(57) ABSTRACT

This disclosure relates to a new crystalline microporous silicate solid, designated CIT-10, comprising a two dimensional layered structure, having an organic interlayer sandwiched between individual crystalline silicate layers. This CIT-10 material can be converted to a pure-silicate of RTH topology, as well as two new of pillared silicate structures, designated CIT-11 and CIT-12. This disclosure characterizes new materials and provides methods of preparing and using these new crystalline microporous solids.

### CROSS REFERENCE TO RELATED APPLICATIONS

This application claims the benefit of priority from U.S. Patent Application No. 62/187,074, filed Jun. 30, 2015, the contents of which are incorporated by reference herein in their entirety for all purposes.

### TECHNICAL FIELD

This disclosure relates to new crystalline microporous silicate solids, which are useful precursors for crystalline microporous silicates having RTH and expanded pore size topologies. This disclosure characterizes new materials and provides methods of preparing these and known crystalline microporous solids.

### BACKGROUND

It is estimated that over 90% of chemical processes use a catalyst, with 80% being a heterogeneous catalyst, with a global demand of $15 to $20 billion per year. Microporous materials (pores less than 2 nm) are an important type of heterogeneous catalyst as they offer shape and size selective environments for catalysis to occur. Additionally, they often exhibit robust hydrothermal stability which allows them to be used under demanding process conditions, such as fluid catalytic cracking. Synthetic aluminosilicate zeolites are produced on a scale 1.7-2 million metric tons per year, and their use as catalysts comprises 27% of the world market for zeolites. As the cost of the catalyst is estimated to be only 0.1% of the cost of the final product, the demand to innovate in this area remains high. There currently exist over 200 known microporous material frameworks, but of these less than 20 have been commercialized and the market is dominated by only five major frameworks. In many applications, there is only a single structure and composition to achieve optimal performance, motivating much of the research directed at creating new materials.

The rate of discovery of new microporous materials has accelerated in recent years due to factors including new organic structure directing agents, the use of fluoride as a mineralizing agent as well as the use of heteroatom in syntheses. Much of this discovery is motivated by the fact that a single framework and composition are normally found to achieve optimal performance in a process. Another synthesis strategy that has received increased attention is that of synthesizing layered silicates that can directly form microporous materials via topotactic condensation, or can be pillared using silyating agents or other metal oxide precursors, forming structures with larger pores (generally larger by 2 tetrahedral atoms) than would be formed via topotactic condensation. The use of pillaring in clay systems provides a useful precedent for such pillaring in layered silicate structures.

In a topotactic condensation, a three-dimensional (3D) framework structure is formed from a two-dimensional (2D) layered silicate by condensation of surface silanol groups (Si—OH), releasing water. Some of the initial framework materials prepared via topotactic condensation were FER, formed from the layered precursor denoted PREFER, and MWW, formed from the layered precursor MCM-22(P). After these pioneering efforts, several additional frameworks have been prepared using topotactic condensation, and they include those provided in Table 1:

TABLE 1
Frameworks prepared by topotactic condensation

| | Layered | |
|---|---|---|
| Framework | Precursor | Reference |
| AST | β-helix-layered | Y. Asakura, et al., Chemistry, 2014, 20, 1893-1900 |
| | silicate | |
| CAS-NSI | EU-19, NU-6(1) | A. J. Blake, et al., J. Chem. Soc. Dalt. Trans., 1988, 2513; B. |
| intermediate | | Marler, et al., Microporous Mesoporous Mater., 2006, 90, 87-101; |
| CDO | PLS-1, RUB-36 | S. Zanardi, et al., Angew. Chem. Int. Ed., Engl., 2004, 43, |
| | | 4933-37. |
| | | B. Marler, et al., Microporous Mesoporous Mater., 2006, 90, |
| | | 87-101; T. Ikeda, et al., Angew. Chem. Int. Ed., Engl., 2004, 43, |
| | | 4892-96 |
| FER | PREFER | L. Schreyeck, et al., Microporous Mater., 1996, 6, 259-271 |
| MTF | HPM-2 | A. Rojas, et al., Chem. Mater., 2014, 26, 1161-69. |
| MWW | MCM-22 | M. E. Leonowicz, et al., Science, 1994, 264, 1910-13 |
| PCR | IPC-4 prepared by | W. J. Roth, et al., Nat. Chem., 2013, 5, 628-33 |
| | disassembly of | |
| | UTL | |
| RRO | RUB-39 | Y. X. Wang, et al., Chem. Mater., 2005, 17, 43-49 |
| RWR | RUB-18 | B. Marler, et al., Microporous Mesoporous Mater., 2005, 83, |
| | | 201-211 |
| SOD | RUB-15 | T. Moteki, et al., J. Am. Chem. Soc., 2008, 130, 15780-81 |

All of these layered materials are formed from only three different two-dimensional layered precursors related to FER, MWW and NSI, and the three-dimensional frameworks are formed from different stacking arrangements of these layers. Additionally, methods have been developed to prepare MFI nanosheets that are a single unit cell thick, however, this material generally is not considered to be a layered zeolite precursor.

The silanol groups of the layered zeolite precursors can also be used to prepare larger pore materials, through a pillaring process. This process normally uses dichlorodimethylsilane or diethoxydimethylsilane to react with the silanol groups to form pillars that are coordinated to two methyl groups (or two hydroxyl groups after calcination). This process is normally carried out in acidic media under hydrothermal conditions. Some of the layered materials that have been pillared include PREFER, MWW(P), PLS-1, MCM-47, RUB-36, RUB-39 and Nu-6(1). Additionally, related strategies to prepare porous materials include delamination or exfoliation. Recently, it has also been shown to be possible to introduce catalytic activity in the pillars. Likewise, the use of pillaring in clay systems has allowed the preparation of a range of useful catalyst and catalytic frameworks.

All of the previously reported layered zeolite precursors are dense layers, that is, they contain no pores (8MR or larger) that are perpendicular to the layers. The MWW layer contains a sinusoidal 10MR channel parallel to the ab-plane, but is still dense as this channel is not perpendicular to the layer. However, the nanosheets of MFI that are single unit cell thick do contain a 10MR perpendicular to the layer; a medium size pore. The present inventors recently reported a method to prepared high-silica heulandite (denoted CIT-8) via topotactic condensation from a layered precursor (denoted CIT-8P), that was prepared using a diquaternary organic structure directing agent (OSDA) in fluoride-mediated syntheses. In that case, the building layer that forms CIT-8 (HEU) was the same as that of RUB-41 (RRO), but was formed from a different stacking of the building layer: AA stacking gave the RRO structure while AA′ stacking (where the A′-layer was related to the A-layer by a 180 degree rotation) gave the HEU structure. CIT-8 was prepared from fluoride-mediated, aluminosilicate inorganic conditions across a relatively narrow composition range. It is interesting to note with this material that the OSDA used was considerably larger than what is normally found in preparing topotactic materials such as piperidines, as well as methyl, ethyl and propyl substituted ammoniums.

### SUMMARY

The present disclosure describes a layered precursor to pure-silica RTH and the conditions to prepare and use this precursor. This layered material, designated CIT-10, can be directly calcined to prepare pure-silica RTH (SSZ-5036) or can be pillared, leading to a new microporous material (denoted CIT-11), that is stable to calcination (calcined material denoted CIT-12). CIT-10 is a layered material composed of a new, fourth type of 2D layer containing an 8MR channel going through the layer (denoted RTH-type layer, 8MR pore dimensions of 5.6×2.5 Å). The discovery of the RTH-type layer adds a fourth group of layered zeolite precursors to the already known FER, MWW and NSI layers, and is the first to contain an 8MR through the 2D layer. Pure-silica RTH is now the sixth known microporous material that can be obtained by both direct synthesis and topotactic condensation.

The first crystalline microporous silicate described herein, designated CIT-10, is characterized by powder X-ray diffraction (XRD) patterns, ²⁹Si-MAS NMR, and RED (rotating electron diffraction) structure analysis as consistent with comprising a two dimensional layered crystalline silicate structure, this layered silicate structure having an organic material, including any one of the OSDAs described in this context, positioned between individual crystalline silicate layers. Specific data supporting these characterizations are provided in the Tables and Figures presented herein. This two dimensional layered structure of CIT-10 is also consistent with the ability of this structure to act as a precursor for the topotactic condensation to a crystalline microporous silicate of RTH topology and for the pillaring using silylating reagents to form larger ring structures, designated CIT-11.

As described herein, in certain embodiments, the crystalline microporous silicate designated CIT-10 comprises an occluded or interlayered organic structure, for example an organic structure directing agent (OSDA). In some embodiments, the OSDA comprises a compound having a structure:

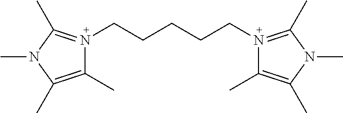

In other embodiments, the crystalline microporous silicate CIT-10 may be converted into a pure silicate of RTH topology by heating the CIT-10 form to at least one temperature in a range of from 300° C. to 800° C.

In still other embodiments, the crystalline microporous silicate CIT-10 may be converted into the second crystalline microporous silicate form described herein, designated CIT-11, by treating the CIT-10 material with a silylating agent under pillaring conditions. In certain of these embodiments, the silylating agent comprises those known to be useful for pillaring such structures, for example including dichlorodimethylsilane and/or diethoxydimethylsilane, and the pillaring conditions comprise the use of strong protic acids and optionally the use of solvents comprising alcohols. Other sources of metal oxide precursors may also be used in such pillaring conditions, giving rise to the corresponding metal oxide or mixed metal oxide pillars between the silicate layers.

The crystalline microporous silicate form designated CIT-11, is characterized by one or more of specific powder X-ray diffraction (XRD) patterns and/or a ²⁹Si-MAS NMR spectrum having characteristic chemical shifts consistent with a pillared structure. The specific characteristic associated with the powder XRD patterns and ²⁹Si-MAS NMR spectra are described herein.

In still other embodiments, as second crystalline microporous silicate form, designated CIT-11, may be calcined to form still another crystalline microporous silicate, designated CIT-12, also having characteristic powder X-ray diffraction (XRD) pattern and ²⁹Si-MAS NMR spectral features. The CIT-12 material may be used as sieves for various chemical separations and may further be treated with various chemical agents, including metallizing agents, to provide catalysts for a range of chemical manipulations. Each of these downstream compositions and uses are contemplated as independent embodiments of the present disclosure.

### BRIEF DESCRIPTION OF THE DRAWINGS

The present application is further understood when read in conjunction with the appended drawings. For the purpose of illustrating the subject matter, there are shown in the drawings exemplary embodiments of the subject matter; however, the presently disclosed subject matter is not limited to the specific methods, processes, devices, and systems disclosed. In addition, the drawings are not necessarily drawn to scale. In the drawings:

FIG. 1 shows representative powder X-ray diffraction (XRD) patterns of CIT-10 (lower) and calcined CIT-10 (pure silica RTH, upper) with selected crystallographic indices

FIGS. 2(A-D) shows SEM images of CIT-10 (FIG. 2A), Si-RTH (FIG. 2B), CIT-11 (FIG. 2C), and CIT-12 (FIG. 2D). Arrowed bars for A, B, and D measure one micron; for C arrow bar measures two microns.

FIG. 3 shows ¹³C NMR of the diquat in D₂O (lower, methanol added as an internal standard), ¹³C CPMAS NMR of CIT-10 showing the occluded diquat (middle) and ¹³C CPMAS NMR of CIT-11 (upper)

FIGS. 4(A-E) shows ²⁹Si MAS NMR of CIT-10 (FIG. 4A), CPMAS NMR of CIT-10 (FIG. 4B), MAS NMR of pure-silica RTH prepared by calcination of CIT-10 (FIG. 4C), MAS NMR of CIT-11 (FIG. 4D), and MAS NMR of CIT-12 (FIG. 4E).

FIG. 5 shows a variable temperature PXRD of CIT-10.

FIG. 6 shows RED (Rotating Electron Diffraction) structure analysis of CIT-10.

FIG. 7 shows TGA data for CIT-10 and CIT-11

FIG. 8 depicts a schematic representation of the topotactic condensation and pillaring of CIT-10.

FIGS. 9(A-D) show PXRD patterns of CIT-10 (FIG. 9A), CIT-11 (FIG. 9B), CIT-12 (FIG. 9C), and pure-silica RTH (FIG. 9D).

FIG. 10 shows representation of an RTH building layer showing 4 independent T atoms. As all 4 have the same multiplicity and only T-1 atoms are on the surface, ideally Q³/Q⁴=¼=0.25.

FIG. 11 shows log plot of argon adsorption isotherms

### DETAILED DESCRIPTION OF ILLUSTRATIVE EMBODIMENTS

The present disclosure contains subject matter related to U.S. patent application Ser. No. 14/602,415 filed Jan. 22, 2015, and describes the synthesis of a new, layered material designated CIT-10 (having two dimensional pure silica layers separated by an organic). Although three other layered materials are known, this is the first layered material that has 8 membered rings running through the plane of the layer, making it a porous layer. CIT-10 can be converted into a number of other microporous materials.

There are three distinct two-dimensional, layered zeolite precursors (FER, MWW and NSI) that can condense through different stacking arrangements of the layers to form various three-dimensional framework materials. These precursors are dense layers in that they do not contain 8-membered ring (MR) or larger pores perpendicular to the two-dimensional layers. This disclosure describes a new material (CIT-10) that consists of a two-dimensional layer that contains an 8MR perpendicular to the layer. Calcination of CIT-10 forms pure-silica RTH (SSZ-50). CIT-10 can be pillared to form a new framework material with a three-dimensional pore system of 8 and 10 MRs, denoted CIT-11, that can be calcined to form a new microporous material denoted CIT-12.

The present invention may be understood more readily by reference to the following description taken in connection with the accompanying Figures and Examples, all of which form a part of this disclosure. It is to be understood that this invention is not limited to the specific products, methods, processes, conditions or parameters described or shown herein, and that the terminology used herein is for the purpose of describing particular embodiments by way of example only and is not intended to be limiting of any claimed invention. Similarly, unless specifically otherwise stated, any description as to a possible mechanism or mode of action or reason for improvement is meant to be illustrative only, and the invention herein is not to be constrained by the correctness or incorrectness of any such suggested mechanism or mode of action or reason for improvement. Throughout this specification, claims, and drawings, it is recognized that the descriptions refer to compositions and processes of making and using said compositions. That is, where the disclosure describes or claims a feature or embodiment associated with a composition or a method of making or using a composition, it is appreciated that such a description or claim is intended to extend these features or embodiment to embodiments in each of these contexts (i.e., compositions, methods of making, and methods of using). Further, where a solid is described as resulting from a given method or process of making, independent embodiments provide a solid composition having the characteristics of the solid so-prepared, but which is not necessarily reliant on that method of making the solid.

Compositions

The present disclosure describes three classes of novel materials. These are described generally in terms of CIT-10, CIT-11, and CIT-12. These labels are used simply for convenience in describing the three types of materials, and should not be construed as limiting the structure in any way. These microporous compositions are described herein as silicate materials, and generally as pure-silicate materials, reflective of the at least two-dimensional silicate layers within each structure. But it should be appreciated that, in the case of CIT-11 and CIT-12, the inclusion of non-silica containing pillars may give rise to structures containing elements beyond silicon and oxygen (as well as the unavoidable impurities derived from the starting materials). Each of these microporous silicate compositions are described herein, as are the methods of making and using the inventive structures.

The various crystalline structures described herein are generally and conveniently described in terms of their characteristic XRD diffraction patterns. Certain embodiments include those structures exhibiting any one of the XRD patterns shown in any one of the Figures of this specification or tabulated peaks. Table 2 provides tabulations of major peaks within each powder XRD pattern, and separate embodiments include those structures having at least the five major peaks of each spectrum, and optionally additional peaks, preferably in order of decreasing relative heights (intensities).

TABLE 2
Representative XRD data for structures described in
this specification; 2-θ values ±0.2 deg

| Calcined pure silica | | | | | | Calcined CIT-11 | |
|---|---|---|---|---|---|---|---|
| RTH | | CIT-10 | | CIT-11 | | (i.e., CIT-12) | |
| | Relative | | Relative | | Relative | | Relative |
| 2-θ | Inten- | 2-θ | Inten- | 2-θ | Inten- | 2-θ | Inten- |
| (deg) | sity | (deg) | sity | (deg) | sity | (deg) | sity |
| 8.73 | 100 | 7.6 | 100 | 6.9 | 100 | 7.7 | 100 |
| 9.18 | 83 | 8.7 | 50 | 8.6 | 46 | 8.8 | 73 |
| 10.28 | 57 | 10.3 | 17 | 10.2 | 31 | 10.3 | 56 |
| 12.62 | 3 | 11.8 | 7 | 15.8 | 2 | 18.1 | 5 |
| 12.99 | 4 | 17.0 | 8 | 17.3 | 4 | 19.3 | 9 |
| 17.87 | 10 | 17.9 | 7 | 18.9 | 8 | 20.7 | 6 |
| 18.92 | 24 | 18.8 | 29 | 20.3 | 4 | 22.6 | 9 |
| 19.89 | 17 | 20.3 | 11 | 21.0 | 3 | 25.6 | 5 |
| 23.33 | 8 | 21.8 | 10 | 22.2 | 4 | 28.5 | 3 |
| 25.25 | 8 | 22.4 | 15 | 25.6 | 2 | 31.1 | 5 |
| 25.77 | 7 | 22.7 | 14 | 28.2 | 1 | | |
| 28.29 | 3 | 22.9 | 15 | 30.8 | 2 | | |
| 30.82 | 4 | 23.6 | 10 | | | | |
| 32.68 | 3 | 24.9 | 7 | | | | |
| | | 28.5 | 8 | | | | |

As described herein, the variation in the scattering angle (two theta) measurements, due to instrument error and to differences between individual samples, is estimated at ±0.15 degrees. Minor variations in the diffraction pattern can result from minor impurities and crystal size; e.g., sufficiently small crystals will affect the shape and intensity of peaks, leading to significant peak broadening. Calcination can also result in changes in the intensities of the peaks as compared to patterns of the “as-made” material, as well as minor shifts in the diffraction pattern. The crystalline solids produced by exchanging the metal or other cations present in the solids with various other cations (NH₄⁺ and then calcining to produce H⁺) yields essentially the same diffraction pattern, although again, there may be minor shifts in the interplanar spacing and variations in the relative intensities of the peaks. Notwithstanding these minor perturbations, the basic crystal lattice remains unchanged by these treatments. Accordingly, the skilled artisan would expect that a description that structures having XRD patterns with peaks within such small variances shown in Table 2 would still be considered within the scope of this invention.

In some embodiments, the crystalline microporous solids may be characterized by the dimensions and directions of the rings (Table 3).

TABLE 3
Representative dimensions of the compositions
described herein (from IZA)

| | 8-MR | 10-MR | 8-MR | 8-MR |
|---|---|---|---|---|
| | [001]^{a} | [100]^{a} | [100]^{a} | [010] |
| CIT-10 | 2.5 × 5.6 Å | N/A | N/A | N/A |
| CIT-11 | 2.5 × 5.6 Å | Yes ^{b} | N/A | Yes ^{b} |
| CIT-12 | 2.5 × 5.6 Å | Yes ^{b} | N/A | Yes ^{b} |
| RTH | 2.5 × 5.6 Å | N/A | 3.8 × 4.1 Å | N/A |

^{a}[001] and [100] refer to crystallographic directions. Such directions are provided for guidance only, and may vary slightly in some embodiments. It is understood that these ring directions are for ideal materials. In real materials, small deviations occur.
^{b} CIT-11 and CIT-12 contain 10-MR along [100] and 8-MR along [010] directions, but the pore dimensions are not known yet as the pillaring process made disorder to the structures and the atomic positions of the structures cannot be solved at this moment. Further, the 10MR and 8MR associated with these materials are expected to have quite flexible pore dimensions due to pillaring.

CIT-10

Certain embodiments provide for crystalline microporous silicates, designated CIT-10. These materials may be characterized at least by one or more of powder X-ray diffraction (XRD) patterns, RED (rotating electron diffraction) structure analyses, and/or ²⁹Si-MAS NMR spectra.

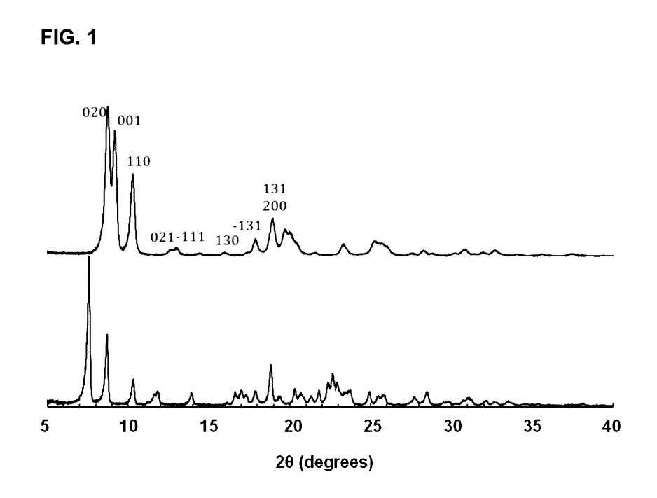

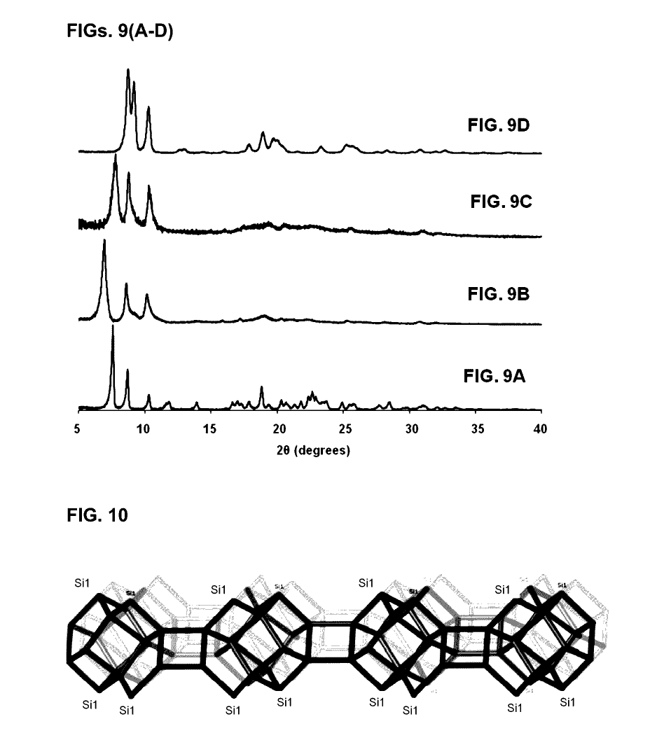

Accordingly, in certain embodiments, the crystalline microporous silicate designated CIT-10 exhibits a powder X-ray diffraction (XRD) pattern exhibiting at least five of the characteristic peaks at 7.6±0.2°, 8.7±0.2°, 10.3±0.2°, 18.8±0.2°, 20.3±0.2°, 21.8±0.2°, 22.4±0.2°, 22.7±0.2°, 22.9±0.2°, and 23.6±0.2° 2-theta. In some independent embodiments, the crystalline microporous silicate exhibits a powder X-ray diffraction (XRD) pattern exhibiting at least 5, 6, 7, 8, 9, or 10 of these characteristic peaks. In other independent embodiments, the crystalline microporous silicate exhibits a powder X-ray diffraction (XRD) pattern exhibiting at least 5, 6, 7, 8, 9, or 10 of the characteristic peaks listed in Table 2. In still other embodiments, the crystalline microporous silicate exhibits a powder X-ray diffraction (XRD) pattern substantially the same as that shown in FIGS. 1 and 9A, allowing for variances in the relative intensities of the peaks.

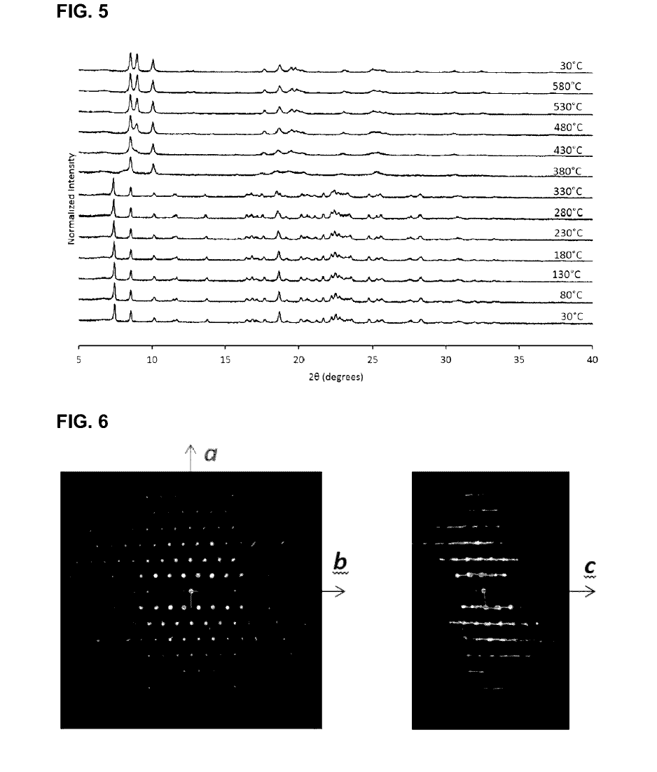

The crystalline microporous silicate designated CIT-10, comprises a crystalline microporous silicate having a two dimensional layered structure, in which an having an organic material, including any one of the OSDAs described herein, is positioned between individual crystalline silicate layers. The specific nature of the organic interlayer is described elsewhere herein. The cationic nature of the OSDA interlayer provides a structure which may be deemed analogous to clay structures in which silicate layers sandwich polycationic inorganic moieties. In addition to the analytical data, this characterization is consistent with the ability of the crystalline microporous silicate to act as a precursor for the topotactic condensation to a crystalline microporous silicate of RTH topology and for the pillaring of such structures using silylating and other reagents as otherwise described herein. For at least these reasons, those embodiments describing the structure of such materials in these terms are considered within the scope of the present disclosure. Further, as shown in FIG. 5, heating this crystalline microporous silicate designated CIT-10 proceeds initially to form a phase before conversion to a pure-silicate RTH phase. This initially formed composition, which may represent a delaminated or a simply layered two-dimensional pure silicate phase, is considered a separate embodiment of the present disclosure.

In other embodiments, the crystalline microporous silicate designated CIT-10, has a structure which is ordered in its a and b directions (i.e., along its two dimensional crystalline layer), but which exhibits some disorder in the c direction (i.e., between layers) as evidenced by RED (rotating electron diffraction) structure analysis. In some embodiments, the crystalline microporous silicate structures exhibits an RED structure analysis such as shown in FIG. 6. Such patterns have also been observed in other two-dimensional crystal forms.

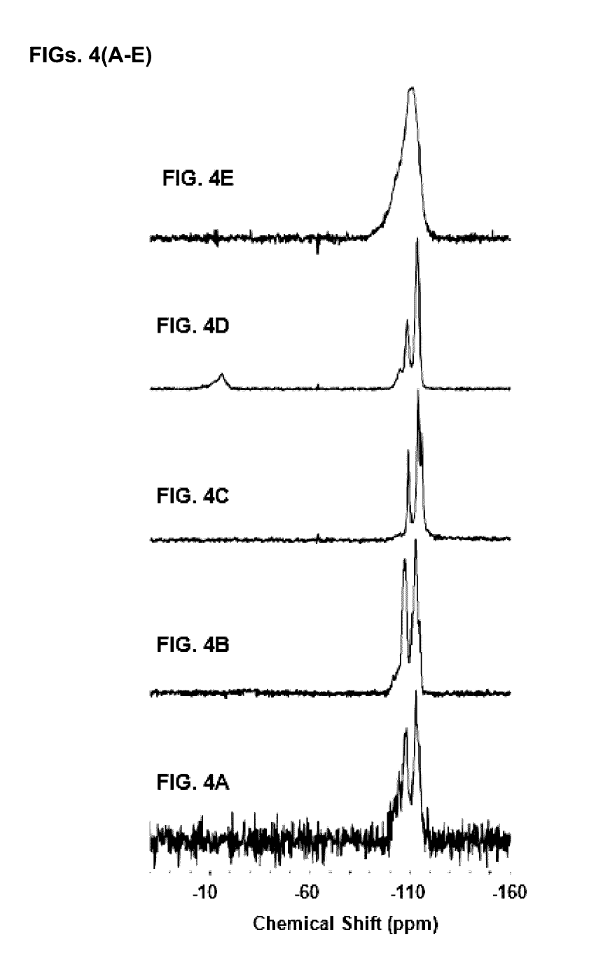

The crystalline microporous silicate designated CIT-10 also exhibits, in some embodiments, a ²⁹Si-MAS NMR spectrum having chemical shifts of −113 ppm, −107 ppm, and −102 ppm, relative to tetramethylsilane (TMS). In some of these embodiments, the chemical shifts of −113 ppm, −107 ppm, and −102 ppm have relative approximate area ratios of 8:5:3. In still other embodiments, the crystalline microporous silicate designated CIT-10 exhibits an ²⁹Si-MAS NMR spectrum substantially similar to that shown in FIGS. 4(A-B). As used herein, in this context, the “substantially similar” refers to a ²⁹Si-MAS NMR spectrum whose resonances exhibit shapes and relative intensities as shown, though it should be appreciated that spectra obtained from larger or smaller magnetic fields than those used herein will provide different shapes and may provide different estimates of intensities. As described elsewhere, the signals at −113 and −107 ppm are assigned to Q⁴ silicon, Si(OSi)₄ coordination, while the signal at −102 ppm is assigned to Q³ silicon, Si(OSi)₃(OH) coordination. The presence of Q³ silicon is expected in a layered material. The ratio of Q³/(Q³+Q⁴) silicon in the as made material is 0.23, which is very close to the theoretical value of 0.25.

As described elsewhere, the crystalline microporous silicate designated CIT-10, comprises an occluded or interlayered organic material. As-prepared, this organic material can be the structure directing agent (OSDA) used in the preparation of the material In some embodiments, the OSDA comprises a diquaternary (dicationic) structure of:

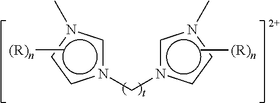

wherein t is 4 or 5, preferably 5; and

R is independently methyl or ethyl, preferably methyl or mainly methyl, and n is independently 1, 2, or 3; said linked pair of quaternary imidazolium cations having associated fluoride or hydroxide ions, preferably substantially free of other halide counterions, i.e., bromide, chloride, or iodide.

In certain preferred embodiments, the linked pair of quaternary imidazolium cations has a structure:

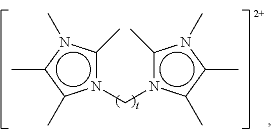

preferably where t is 5; and the associated ions are preferably hydroxide. In some cases, the associated ion is fluoride ion. If initially prepared using fluoride ion, these ions may be removed by any appropriate ion exchange technique.

In specific embodiments, the OSDA comprises a compound having a structure:

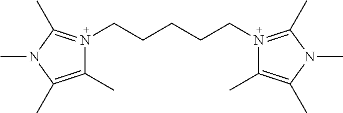

As used herein, the term “linked pair of quaternary imidazolium cations” is intended to connote that two quaternary imidazolium cations are linked by the carbon linker, and not that the two quaternized cations are necessarily identical, though this is preferred.

The CIT-10 is prepared by hydrothermally treating a composition comprising a silicate source, and a mineralizing agent, in the presence of an OSDA under conditions sufficient to form the desired crystalline product, and optionally recovering and further processing the crystalline products.

The counterions of the organic structure directing agents can be fluoride or hydroxide, and substantially free of other halide counterions, i.e., bromide, chloride, or iodide. In this context, the term “substantially free” refers to a condition where no bromide, chloride, or iodide are added to the composition or process, and in fact, reasonable efforts are taken to remove these from the composition or process, e.g., by ion exchange methods. It does not require absolute absence of these anions, as for example, as may result from incidental residual bromide, chloride, or iodide contained within the inorganic materials.

Typical sources of silicon oxide for the reaction mixtures include alkoxides, hydroxides, or oxides of silicon, or combination thereof. Exemplary compounds also include silicates (including sodium silicate), silica hydrogel, silicic acid, fumed silica, colloidal silica, tetra-alkyl orthosilicates, and silica hydroxides.

In processing the crystalline microporous solids, the reaction mixture is maintained at an elevated temperature until the crystals of the desired product form are formed. The hydrothermal crystallization is usually conducted under autogenous pressure, at a temperature between 100° C. to about 200° C., preferably about 140° C. to about 180° C. or from about 160° C. to about 180° C., for a time effective for crystallizing the desired crystalline microporous solid. The crystallization period is typically greater than I day and preferably from about 1 day to about 40 days, or from about 3 days to about 20 days. Preferably, the silicate is prepared using mild stirring or agitation.

During the hydrothermal crystallization step, the crystalline microporous solids can be allowed to nucleate spontaneously from the reaction mixture. The use of product crystals as seed material can be advantageous in decreasing the time necessary for complete crystallization to occur. In addition, seeding can lead to an increased purity of the product obtained by promoting the nucleation and/or formation of the desired crystalline microporous solid over any undesired phases. When used as seeds, such seed crystals are added in an amount between 0.1 and 5% or between 0.1 and 10% of the weight of silicate-source used in the reaction mixture.

Once the crystals have formed, the solid product can be separated from the reaction mixture by standard mechanical separation techniques such as filtration or centrifugation. The crystals can be water-washed and then dried, e.g., at 90° C. to 150° C. for 8 to 24 hours, to obtain the as-synthesized crystalline microporous solids. The drying step can be performed at atmospheric pressure or under vacuum.

In various embodiments, the processes described herein produce or are capable of producing compositionally “clean” crystalline microporous materials. That is, in various embodiments, the crystalline microporous materials described herein are at least 75%, 80%, 85%, 90%, 95%, or 98% by weight of the nominal topology. In some embodiments, the crystalline microporous materials exhibit XRD patterns where other crystalline topologies are undetectable.

Additional embodiments include those process comprising heating the crystalline microporous silicate designated CIT-10 to at least one temperature in a range of from 300° C. to 800° C. for a time sufficient to provide a crystalline microporous silicate of RTH topology. In certain independent embodiment, the at least one temperature may be at least one temperature defined by one or more of the ranges from 380° C. to 430° C., from 430° C. to 480° C., from 480° C. to 530° C., from 530° C. to 580° C., from 580° C. to 620° C., from 620° C. to 660° C., from 660° C. to 700° C., from 700° C. to 750° C., or from 750° C. to 800° C. These temperatures are applied for a time sufficient to remove any occluded organic material from between the silicate layers; e.g., 6-12 hours at these temperatures. Upon calcination, the ²⁹Si MAS NMR spectrum no longer shows the presence of any Q³ silicon and instead shows 3 resonances in the Q⁴ region at −116, −114 and −109 ppm, with area ratios of 1:2:1. These area ratios agree with the crystal structure of RTH, as it contains 4 independent T-sites.

CIT-11

A second class of crystalline microporous silicates may be obtained by the application of conditions consistent with pillaring to CIT-10. In some cases, this second class of crystalline microporous silicates may be prepared by reacting the crystalline microporous silicate designated CIT-10 with a silylating agent, or other metal oxide precursor, in the presence of a strong acid and optionally an alcohol under conditions sufficient to effect the desired transformation. In certain embodiments, this includes the strong acid is or comprises nitric or hydrochloric acid, preferably hydrochloric acid, in a concentration ranging from about 1 M to about 1.5 M, preferably 1.25 M. In some embodiments, the alcohol is methanol, ethanol, or propanol, or a combination thereof, preferably ethanol. In certain embodiments, the reaction conditions include contacting the CIT-10 with the silylating agent in the presence of a strong acid and an alcohol in at least one temperature in a range of from about 120° C. to about 225° C., preferably 175° C., under autogenous pressures. Depending on the temperature and other parameters, the time sufficient to effect the transformation can typically be 12 to 72 hours, preferably about 24 to 48 hours, or about 24 hours. In specific embodiments, the silylating agent comprises those known to be useful for pillaring such structures, for example including dichlorodimethylsilane and/or diethoxydimethylsilane. In certain other embodiments, other metal oxide precursors may also be used in this capacity, under any of the conditions just described or known to be useful in pillaring clays. As described elsewhere herein, such metal oxide precursors may also include a source of aluminum oxide, boron oxide, chromium oxide, gallium oxide, iron oxide, nickel oxide, titanic, tin oxide, vanadia, zinc oxide, zirconium oxide, or mixture thereof (e.g., mixed oxides of Cr/Al, Fe/Al, Ga/Al, Si/Al. Zr/Al). Such precursors are known in the art of zeolite and clay chemistries. See, references cited elsewhere herein, including, e.g., Trees De Baerdemaeker, et al., “A new class of solid Lewis acid catalysts based on interlayer expansion of layered silicates of the RUB-36 type with heteroatoms,” J. Mater. Chem. A, 2014, 2, 9709-9717, which describes the use of an Fe salt instead of a silylating agent was used to incorporate iron oxide in the linking sites in between the layers.

Independent of the methods used to prepare these materials, other embodiments provide crystalline microporous silicates, designated CIT-11. These crystalline microporous silicates exhibit a powder X-ray diffraction (XRD) pattern exhibiting at least five of the characteristic peaks at 6.9±0.2°, 8.6±0.2°, 10.2, ±0.2°, 15.8±0.2°, 17.3±0.2°, 18.9±0.2°, 20.3±0.2°, 21.0±0.2°, 22.2±0.2°, 25.6±0.2°, and 30.8±0.2° 2-theta. In some independent embodiments, the crystalline microporous silicate exhibits a powder X-ray diffraction (XRD) pattern exhibiting at least 5, 6, 7, 8, 9, or 10 of these characteristic peaks. In other independent Aspects of this Embodiment, the crystalline microporous silicate exhibits a powder X-ray diffraction (XRD) pattern exhibiting at least 5, 6, 7, 8, 9, or 10 of the characteristic peaks listed in Table 2. In some embodiments, the crystalline microporous silicate exhibits a powder X-ray diffraction (XRD) pattern substantially the same as that shown in FIG. 9B, allowing for variances in the relative intensities of the peaks. Where the pillars comprise other metal oxides than silicates, slight and predictable changes in the specific powder XRD patterns may be observed.

As with the crystalline microporous silicates, designated CIT-10, the crystalline microporous silicates designated CIT-11 exhibit unique ²⁹Si-MAS NMR spectra. For pure-silica materials, these ²⁹Si-MAS NMR spectra exhibit chemical shifts of −113.5 ppm, −108.4 ppm, −104.5 ppm, and −15.3 ppm, relative to tetramethylsilane (TMS). In some of these embodiments, the chemical shifts of −113.5 ppm, −108.4 ppm, −104.5 ppm, and −15.3 ppm have relative approximate area ratios of 20:8:2:5. In other embodiments, the crystalline microporous silicate exhibits a ²⁹Si-MAS NMR spectrum substantially similar to that shown in FIG. 4D. As discussed elsewhere, the resonances at −113.5 and −108.4 are assigned to Q⁴ silicon and the resonance at −104.5 is assigned to residual Q³ silicon. The Q³/(Q³+Q⁴) ratio in the pillared material is 0.07, a significant decrease from 0.23 in CIT-10, indicating that a substantial amount of Q³ species have been consumed in linking the layers of the material. The resonance at −15.3 is assigned to bridging silanol groups bonded to two methyl groups, that is Si(CH₃)₂(OSi)₂ coordination. The ratio of (Q²+Q³)/(Q²+Q³+Q⁴)) is 0.25, consistent with the expected value from the RTH layer. Where the pillars comprise other metal oxides other than silica, the ²⁹Si-MAS NMR spectra will obviously reflect these differences.

CIT-12

A third class of crystalline microporous silicates, designated CIT-12, may be obtained by the calcining the crystalline microporous silicates, designated CIT-11. Such calcining conditions may comprising heating the CIT-11 materials to at least one temperature in a range of from 300° C. to 800° C. for a time sufficient to provide a CIT-12 material. In certain independent embodiment, the at least one temperature may be at least one temperature defined by one or more of the ranges from 380° C. to 430° C., from 430° C. to 480° C., from 480° C. to 530° C., from 530° C. to 580° C., from 580° C. to 620° C., from 620° C. to 660° C., from 660° C. to 700° C., from 700° C. to 750° C., or from 750° C. to 800° C.

Independent of their method of preparation, pure silicate versions of CIT-12, exhibit powder X-ray diffraction (XRD) patterns exhibiting at least five of the characteristic peaks at 7.7±0.2°, 8.8±0.2°, 10.3±0.2°, 18.1±0.2°, 19.3±0.2°, 20.7±0.2°, 22.6±0.2°, 25.6±0.2°, 28.5±0.2°, and 31.1±0.2° 2-theta. In some independent embodiments, these crystalline microporous silicate exhibits a powder X-ray diffraction (XRD) pattern exhibiting at least 5, 6, 7, 8, 9, or 10 of these characteristic peaks. In other independent embodiments, these crystalline microporous silicate exhibits a powder X-ray diffraction (XRD) pattern exhibiting at least 5, 6, 7, 8, 9, or 10 of the characteristic peaks listed in Table 2. In some embodiments, the crystalline microporous silicate exhibits a powder X-ray diffraction (XRD) pattern substantially the same as that shown in FIG. 9C, allowing for variances in the relative intensities of the peaks. Again, where the pillars comprise other metal oxides than silicates, slight and predictable changes in the specific powder XRD patterns may be observed.

The crystalline microporous pure silicates of the CIT-12 class exhibit a broad resonance in an ²⁹Si-MAS NMR spectrum at chemical shifts of about −110 ppm relative to tetramethylsilane (TMS). In certain embodiments, the crystalline microporous silicate exhibits an ²⁹Si-MAS NMR spectrum substantially similar to that shown in FIG. 4E. Such a single broad resonance has been observed in other pillared, calcined materials such as PLS-4. No obvious peak for Q² silicon can be seen near −90 ppm, but may be obscured by the much broader resonance. Again, where the pillars comprise other metal oxides other than silica, the ²⁹Si-MAS NMR spectra will obviously reflect these differences.

Further Processing

Any of the materials described herein, including the CIT-10, CIT-11, and CIT-12 compositions, may be further processed to provide catalytic materials. In certain embodiments one or more of these materials may be treated with an aqueous alkali, alkaline earth, transition metal, rare earth metal, ammonium or alkylammonium salt, as described elsewhere herein; and/or treated with at least one type of transition metal or transition metal oxide, as described elsewhere herein. In specific independent embodiments, the CIT-12 materials are so treated.

It is often desirable to remove any alkali metal cation by ion exchange and replace it with hydrogen, ammonium, or any desired metal ion. The crystalline microporous pure silicates can also be steamed; steaming helps stabilize the crystalline lattice to attack from acids. Alternatively, or additionally, the calcined materials may be treated with aqueous ammonium salts (e.g., NH₄NO₃) to remove any residual inorganic cations in the pores of the crystalline solid.

The crystalline microporous pure silicates can be used in intimate combination with hydrogenating components, such as tungsten, vanadium molybdenum, rhenium, nickel cobalt, chromium, manganese, or a noble metal, such as palladium or platinum, for those applications in which a hydrogenation-dehydrogenation function is desired.

Metals may also be introduced into the crystalline microporous solid by replacing some of the cations in the crystalline microporous solid with metal cations via standard ion exchange techniques. (see, for example, U.S. Pat. No. 3,140,249 issued Jul. 7, 1964 to Plank et al.; U.S. Pat. No. 3,140,251 issued Jul. 7, 1964 to Plank et al.; and U.S. Pat. No. 3,140,253 issued Jul. 7, 1964 to Plank et al.). Typical replacing cations can include metal cations, e.g., rare earth, Group 1, Group 2 and Group 8 metals, as well as their mixtures. Cations of metals such as rare earth, Mn, Ca, Mg, Zn, Cd, Pt, Pd, Ni, Co, Ti, Al, Sn, and Fe are particularly preferred.

Following contact with the salt solution of the desired replacing cation, the crystalline microporous solid is typically washed with water and dried at temperatures ranging from 65° C. to about 200° C. After washing, the crystalline microporous solid can be calcined in air or inert gas at temperatures ranging from about 25° C. to about 200° C. or from about 200° C. to about 850° C. or about 1000° C., as described above and depending on the nature of the calcining atmosphere, for periods of time ranging from 1 to 48 hours or more, to produce a catalytically active product especially useful in hydrocarbon conversion processes. Regardless of the cations present in the synthesized form of the crystalline microporous solid, the spatial arrangement of the atoms which form the basic crystal lattice of the crystalline solid remains essentially unchanged.

The crystalline microporous solids may also be treated under conditions so as to incorporate at least one type of transition metal or transition metal oxide catalyst into the pore structure, for example by vapor or chemical deposition or precipitation. As used herein, the term “transition metal” refers to any element in the d-block of the periodic table, which includes groups 3 to 12 on the periodic table. In actual practice, the f-block lanthanide and actinide series are also considered transition metals and are called “inner transition metals. Scandium, yttrium, titanium, zirconium, vanadium, manganese, chromium, molybdenum, tungsten, iron, ruthenium, osmium, cobalt, rhodium, iridium, nickel, palladium, platinum, copper, silver, gold, or mixtures thereof are preferred.

The as-synthesized or calcined crystalline microporous solids can be formed into a wide variety of physical shapes. Generally speaking, the crystalline microporous pure solids can be in the form of a powder, a granule, or a molded product, such as extrudate. In cases where the catalyst is molded, such as by extrusion with an organic binder, these crystalline solids can be extruded before drying, or, dried or partially dried and then extruded. The crystalline microporous solids can be composited with other materials resistant to the temperatures and other conditions employed in organic conversion processes. Such matrix materials include active and inactive materials and synthetic or naturally occurring crystalline solids, including zeolites, as well as inorganic materials such as clays, silica and metal oxides.

Use of the Inventive Compositions—Catalysis

In various embodiments, the crystalline microporous silicate solids of the present invention, calcined, doped, or treated with the catalysts described herein, may mediate or catalyze an array of chemical transformation. Such transformations may include carbonylating DME with CO at low temperatures, reducing NOx with methane (e.g., in exhaust applications) or an olefin in the presence of oxygen, cracking, hydrocracking, dehydrogenating, converting paraffins to aromatics, MTO, isomerizing aromatics (e.g., xylenes), disproportionating aromatics (e.g., toluene), alkylating aromatic hydrocarbons, oligomerizing alkenes, aminating lower alcohols, separating and sorbing lower alkanes, hydrocracking a hydrocarbon, dewaxing a hydrocarbon feedstock, isomerizing an olefin, producing a higher molecular weight hydrocarbon from lower molecular weight hydrocarbon, reforming a hydrocarbon, converting lower alcohol or other oxygenated hydrocarbons to produce olefin products, epoxiding olefins with hydrogen peroxide, reducing the content of an oxide of nitrogen contained in a gas stream in the presence of oxygen, or separating nitrogen from a nitrogen-containing gas mixture by contacting the respective feedstock with the a catalyst comprising the crystalline microporous solid of any one of materials described herein under conditions sufficient to affect the named transformation. Particularly attractive applications include in which these silicates are expected to be useful include catalytic cracking, hydrocracking, dewaxing, alkylation, and olefin and aromatics formation reactions. Additional applications include gas drying and separation.

Specific embodiments provide hydrocracking processes, each process comprising contacting a hydrocarbon feedstock under hydrocracking conditions with a catalyst comprising a crystalline microporous solid of this invention, preferably predominantly in the hydrogen form.

Still other embodiments provide processes for dewaxing hydrocarbon feedstocks, each process comprising contacting a hydrocarbon feedstock under dewaxing conditions with a catalyst comprising a crystalline microporous solid of this invention. Yet other embodiments provide processes for improving the viscosity index of a dewaxed product of waxy hydrocarbon feeds, each process comprising contacting the waxy hydrocarbon feed under isomerization dewaxing conditions with a catalyst comprising a crystalline microporous solid of this invention.

Additional embodiments include those process for producing a C20+ lube oil from a C20+ olefin feed, each process comprising isomerizing said olefin feed under isomerization conditions over a catalyst comprising at least one transition metal catalyst and a crystalline microporous solid of this invention.

Also included in the present invention are processes for isomerization dewaxing a raffinate, each process comprising contacting said raffinate, for example a bright stock, in the presence of added hydrogen with a catalyst comprising at least one transition metal and a crystalline microporous solid of this invention.

Other embodiments provide for dewaxing a hydrocarbon oil feedstock boiling above about 350° F. and containing straight chain and slightly branched chain hydrocarbons comprising contacting said hydrocarbon oil feedstock in the presence of added hydrogen gas at a hydrogen pressure of about 15-3000 psi with a catalyst comprising at least one transition metal and a crystalline microporous solid of this invention, preferably predominantly in the hydrogen form.

Also included in the present invention is a process for preparing a lubricating oil which comprises hydrocracking in a hydrocracking zone a hydrocarbonaceous feedstock to obtain an effluent comprising a hydrocracked oil, and catalytically dewaxing said effluent comprising hydrocracked oil at a temperature of at least about 400° F. and at a pressure of from about 15 psig to about 3000 psig in the presence of added hydrogen gas with a catalyst comprising at least one transition metal and a crystalline microporous solid of this invention.

Also included in this invention is a process for increasing the octane of a hydrocarbon feedstock to produce a product having an increased aromatics content, each process comprising contacting a hydrocarbonaceous feedstock which comprises normal and slightly branched hydrocarbons having a boiling range above about 40° C. and less than about 200° C., under aromatic conversion conditions with a catalyst comprising a crystalline microporous solid of this invention. In these embodiments, the crystalline microporous solid is preferably made substantially free of acidity by neutralizing said solid with a basic metal. Also provided in this invention is such a process wherein the crystalline microporous solid contains a transition metal component.

Also provided by the present invention are catalytic cracking processes, each process comprising contacting a hydrocarbon feedstock in a reaction zone under catalytic cracking conditions in the absence of added hydrogen with a catalyst comprising a crystalline microporous solid of this invention. Also included in this invention is such a catalytic cracking process wherein the catalyst additionally comprises an additional large pore crystalline cracking component.

This invention further provides isomerization processes for isomerizing C4 to C7 hydrocarbons, each process comprising contacting a feed having normal and slightly branched C4 to C hydrocarbons under isomerizing conditions with a catalyst comprising a crystalline microporous solid of this invention, preferably predominantly in the hydrogen form. The crystalline microporous solid may be impregnated with at least one transition metal, preferably platinum. The catalyst may be calcined in a steam/air mixture at an elevated temperature after impregnation of the transition metal.

Also provided by the present invention are processes for alkylating an aromatic hydrocarbon, each process comprising contacting under alkylation conditions at least a molar excess of an aromatic hydrocarbon with a C2 to C20 olefin under at least partial liquid phase conditions and in the presence of a catalyst comprising a crystalline microporous solid of this invention, preferably predominantly in the hydrogen form. The olefin may be a C2 to C4 olefin, and the aromatic hydrocarbon and olefin may be present in a molar ratio of about 4:1 to about 20:1, respectively. The aromatic hydrocarbon may be selected from the group consisting of benzene, toluene, ethylbenzene, xylene, or mixtures thereof.

Further provided in accordance with this invention are processes for transalkylating an aromatic hydrocarbon, each of which process comprises contacting under transalkylating conditions an aromatic hydrocarbon with a polyalkyl aromatic hydrocarbon under at least partial liquid phase conditions and in the presence of a catalyst comprising a crystalline microporous solid of this invention, preferably predominantly in the hydrogen form. The aromatic hydrocarbon and the polyalkyl aromatic hydrocarbon may be present in a molar ratio of from about 1:1 to about 25:1, respectively. The aromatic hydrocarbon may be selected from the group consisting of benzene, toluene, ethylbenzene, xylene, or mixtures thereof, and the polyalkyl aromatic hydrocarbon may be a dialkylbenzene.

Further provided by this invention are processes to convert paraffins to aromatics, each of which process comprises contacting paraffins under conditions which cause paraffins to convert to aromatics with a catalyst comprising a crystalline microporous solid of this invention, said catalyst comprising gallium, zinc, or a compound of gallium or zinc.

In accordance with this invention there is also provided processes for isomerizing olefins, each process comprising contacting said olefin under conditions which cause isomerization of the olefin with a catalyst comprising a crystalline microporous solid of this invention.

Further provided in accordance with this invention are processes for isomerizing an isomerization feed, each process comprising an aromatic C8 stream of xylene isomers or mixtures of xylene isomers and ethylbenzene, wherein a more nearly equilibrium ratio of ortho-, meta- and para-xylenes is obtained, said process comprising contacting said feed under isomerization conditions with a catalyst comprising a crystalline microporous solid of this invention.

The present invention further provides processes for oligomerizing olefins, each process comprising contacting an olefin feed under oligomerization conditions with a catalyst comprising a crystalline microporous solid of this invention.

This invention also provides processes for converting lower alcohols and other oxygenated hydrocarbons, each process comprising contacting said lower alcohol (for example, methanol, ethanol, or propanol) or other oxygenated hydrocarbon with a catalyst comprising a crystalline microporous solid of this invention under conditions to produce liquid products.

Also provided by the present invention are processes for reducing oxides of nitrogen contained in a gas stream in the presence of oxygen wherein each process comprises contacting the gas stream with a crystalline microporous solid of this invention. The a crystalline microporous solid may contain a metal or metal ions (such as cobalt, copper or mixtures thereof) capable of catalyzing the reduction of the oxides of nitrogen, and may be conducted in the presence of a stoichiometric excess of oxygen. In a preferred embodiment, the gas stream is the exhaust stream of an internal combustion engine.

Also provided are processes for converting synthesis gas containing hydrogen and carbon monoxide, also referred to as syngas or synthesis gas, to liquid hydrocarbon fuels, using a catalyst comprising any of the silicates described herein, including those having CIT-12 frameworks, and Fischer-Tropsch catalysts. Such catalysts are described in U.S. Pat. No. 9,278,344, which is incorporated by reference for its teaching of the catalysts and methods of using the catalysts. The Fischer-Tropsch component includes a transition metal component of groups 8-10 (i.e., Fe, Ru, Os, Co, Rh, IR, Ni, Pd, Pt), preferably cobalt, iron and/or ruthenium. The optimum amount of catalytically active metal present depends inter alia on the specific catalytically active metal. Typically, the amount of cobalt present in the catalyst may range from 1 to 100 parts by weight per 100 parts by weight of support material, preferably from 10 to 50 parts by weight per 100 parts by weight of support material. In one embodiment, from 15 to 45 wt % cobalt is deposited on the hybrid support as the Fischer-Tropsch component. In another embodiment from 20 to 45 wt % cobalt is deposited on the hybrid support. The catalytically active Fischer-Tropsch component may be present in the catalyst together with one or more metal promoters or co-catalysts. The promoters may be present as metals or as metal oxide, depending upon the particular promoter concerned. Suitable promoters include metals or oxides of transition metals, including lanthanides and/or the actinides or oxides of the lanthanides and/or the actinides. As an alternative or in addition to the metal oxide promoter, the catalyst may comprise a metal promoter selected from Groups 7 (Mn, Tc, Re) and/or Groups 8-10. In some embodiments, the Fischer-Tropsch component further comprises a cobalt reduction promoter selected from the group consisting of platinum, ruthenium, rhenium, silver and combinations thereof. The method employed to deposit the Fischer-Tropsch component on the hybrid support involves an impregnation technique using aqueous or non-aqueous solution containing a soluble cobalt salt and, if desired, a soluble promoter metal salt, e.g., platinum salt, in order to achieve the necessary metal loading and distribution required to provide a highly selective and active hybrid synthesis gas conversion catalyst.

Still further process embodiments include those for reducing halide concentration in an initial hydrocarbon product comprising undesirable levels of an organic halide, the process comprising contacting at least a portion of the hydrocarbon product with a composition comprising any of the silicate structures described herein, including CIT-12, under organic halide absorption conditions to reduce the halogen concentration in the hydrocarbon. The initial hydrocarbon product may be made by a hydrocarbon conversion process using an ionic liquid catalyst comprising a halogen-containing acidic ionic liquid. In some embodiments, the organic halide content in the initial hydrocarbon product is in a range of from 50 to 4000 ppm; in other embodiments, the halogen concentrations are reduced to provide a product having less than 40 ppm. In other embodiments, the production may realize a reduction of 85%, 90%, 95%, 97%, or more. The initial hydrocarbon stream may comprise an alkylate or gasoline alkylate. Preferably the hydrocarbon alkylate or alkylate gasoline product is not degraded during the contacting. Any of the materials or process conditions described in U.S. Pat. No. 8,105,481 are considered to describe the range of materials and process conditions of the present invention. U.S. Pat. No. 8,105,481 is incorporated by reference at least for its teachings of the methods and materials used to effect such transformations (both alkylations and halogen reductions).

Still further process embodiments include those processes for increasing the octane of a hydrocarbon feedstock to produce a product having an increased aromatics content comprising contacting a hydrocarbonaceous feedstock which comprises normal and slightly branched hydrocarbons having a boiling range above about 40 C and less than about 200 C under aromatic conversion conditions with the catalyst.

Specific conditions for many of these transformations are known to those of ordinary skill in the art. Exemplary conditions for such reactions/transformations may also be found in WO/1999/008961, U.S. Pat. Nos. 4,544,538, 7,083,714, 6,841,063, and 6,827,843, each of which are incorporated by reference herein in its entirety for at least these purposes.

Depending upon the type of reaction which is catalyzed, the microporous solid may be predominantly in the hydrogen form, partially acidic or substantially free of acidity. The skilled artisan would be able to define these conditions without undue effort. As used herein, “predominantly in the hydrogen form” means that, after calcination (which may also include exchange of the pre-calcined material with NH₄⁺ prior to calcination), at least 80% of the cation sites are occupied by hydrogen ions and/or rare earth ions.

Use of the Inventive Compositions—Other

The silicates of the present invention may also be used as adsorbents for gas separations. For example, these silicate can also be used as hydrocarbon traps, for example, as a cold start hydrocarbon trap in combustion engine pollution control systems. In particular, such silicate may be particularly useful for trapping C₃ fragments. Such embodiments may comprise processes and devices for trapping low molecular weight hydrocarbons from an incoming gas stream, the process comprising passing the gas stream across or through a composition comprising any one of the crystalline microporous silicate compositions described herein, so as to provide an outgoing gas stream having a reduced concentration of low molecular weight hydrocarbons relative to the incoming gas stream. In this context, the term “low molecular weight hydrocarbons” refers to C1-C6 hydrocarbons or hydrocarbon fragments.

The silicates of the present invention may also be used in a process for treating a cold-start engine exhaust gas stream containing hydrocarbons and other pollutants, wherein the process comprises or consist of flowing the engine exhaust gas stream over one of the silicate compositions of the present invention which preferentially adsorbs the hydrocarbons over water to provide a first exhaust stream, and flowing the first exhaust gas stream over a catalyst to convert any residual hydrocarbons and other pollutants contained in the first exhaust gas stream to innocuous products and provide a treated exhaust stream and discharging the treated exhaust stream into the atmosphere.

The silicates of the present invention can also be used to separate gases. For example, these can be used to separate water, carbon dioxide, and sulfur dioxide from fluid streams, such as low-grade natural gas streams, and carbon dioxide from natural gas. The compositions described herein, especially the CIT-12 compositions, at least by analogy to their pillared clay analogs, are also seen to be useful in other applications including removal of H₂O, CO₂ and SO₂ from fluid streams, such as low-grade natural gas streams, and separations of gases, including noble gases, N₂, O₂, fluorochemicals and formaldehyde). Exemplary applications will be apparent to the skilled person upon a reading of the present disclosure. Typically, the molecular sieve is used as a component in a membrane that is used to separate the gases. Examples of such membranes are disclosed in U.S. Pat. No. 6,508,860.

For each of the preceding processes described, additional corresponding embodiments include those comprising a device or system comprising or containing the materials described for each process. For example, in the gas of the gas trapping, additional embodiments include those devices known in the art as hydrocarbon traps which may be positioned in the exhaust gas passage of a vehicle. In such devices, hydrocarbons are adsorbed on the trap and stored until the engine and exhaust reach a sufficient temperature for desorption. The devices may also comprise membranes comprising the silicate compositions, useful in the processes described.

These crystalline microporous silicates may also be incorporated into polymer-composite membranes by known methods, the polymers comprising, for example, polyimide, polyethersulfone, polyetheretherketone, and mixtures and copolymers thereof. In other embodiments, the LTA compositions, as supported films or membranes, may be used as reaction templates, separation media, or dielectrics.

Terms

In the present disclosure the singular forms “a,” “an,” and “the” include the plural reference, and reference to a particular numerical value includes at least that particular value, unless the context clearly indicates otherwise. Thus, for example, a reference to “a material” is a reference to at least one of such materials and equivalents thereof known to those skilled in the art, and so forth.

When a value is expressed as an approximation by use of the descriptor “about,” it will be understood that the particular value forms another embodiment. In general, use of the term “about” indicates approximations that can vary depending on the desired properties sought to be obtained by the disclosed subject matter and is to be interpreted in the specific context in which it is used, based on its function. The person skilled in the art will be able to interpret this as a matter of routine. In some cases, the number of significant figures used for a particular value may be one non-limiting method of determining the extent of the word “about.” In other cases, the gradations used in a series of values may be used to determine the intended range available to the term “about” for each value. Where present, all ranges are inclusive and combinable. That is, references to values stated in ranges include every value within that range.

It is to be appreciated that certain features of the invention which are, for clarity, described herein in the context of separate embodiments, may also be provided in combination in a single embodiment. That is, unless obviously incompatible or specifically excluded, each individual embodiment is deemed to be combinable with any other embodiment(s) and such a combination is considered to be another embodiment. Conversely, various features of the invention that are, for brevity, described in the context of a single embodiment, may also be provided separately or in any sub-combination. Finally, while an embodiment may be described as part of a series of steps or part of a more general structure, each said step may also be considered an independent embodiment in itself, combinable with others.

The transitional terms “comprising,” “consisting essentially of,” and “consisting” are intended to connote their generally in accepted meanings in the patent vernacular; that is, (i) “comprising,” which is synonymous with “including,” “containing,” or “characterized by,” is inclusive or open-ended and does not exclude additional, unrecited elements or method or process steps; (ii) “consisting of” excludes any element, step, or ingredient not specified in the claim; and (iii) “consisting essentially of” limits the scope of a claim to the specified materials or steps “and those that do not materially affect the basic and novel characteristic(s)” of the claimed invention. Embodiments described in terms of the phrase “comprising” (or its equivalents), also provide, as embodiments, those which are independently described in terms of “consisting of” and “consisting essentially of” For those embodiments provided in terms of “consisting essentially of,” the basic and novel characteristic(s) of a process is the ability to provide a microporous material having the designated topologies, and of a product or intermediate, one having the designated topology.

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are described herein.

Throughout this specification, words are to be afforded their normal meaning, as would be understood by those skilled in the relevant art. However, so as to avoid misunderstanding, the meanings of certain terms will be specifically defined or clarified.

“Lower alcohols” or lower alkanes refer to alcohols or alkanes, respectively, having 1-10 carbons, linear or branched, preferably 1-6 carbon atoms and preferably linear. Methanol, ethanol, propanol, butanol, pentanol, and hexanol are examples of lower alcohols. Methane, ethane, propane, butane, pentane, and hexane are examples of lower alkanes.

Unless otherwise indicated, the term “isolated” means physically separated from the other components so as to be free of solvents or other impurities; additional embodiments include those where the compound is substantially the only solute in a solvent or solvent fraction, such a analytically separated in a liquid or gas chromatography phase.

The terms “method(s)” and “process(es)” are considered interchangeable within this disclosure.

The term “microporous,” according to IUPAC notation refers to a material having pore diameters of less than 2 nm. Similarly, the term “macroporous” refers to materials having pore diameters of greater than 50 nm. And the term “mesoporous” refers to materials whose pore sizes are intermediate between microporous and macroporous. Within the context of the present disclosure, the material properties and applications depend on the properties of the framework such as pore size and dimensionality, cage dimensions and material composition. Due to this there is often only a single framework and composition that gives optimal performance in a desired application.

“Optional” or “optionally” means that the subsequently described circumstance may or may not occur, so that the description includes embodiments where the circumstance occurs and instances where it does not. For example, the phrase “optionally substituted” means that a non-hydrogen substituent may or may not be present on a given atom, and, thus, the description includes structures wherein a non-hydrogen substituent is present and structures wherein a non-hydrogen substituent is not present. Similarly, the phrase “optionally isolated” means that the target material may or may not be separated from other materials used or generated in the method, and, thus, the description includes separate embodiments where the target molecule or other material is separated and where the target material is not separated, such that subsequence steps are conducted on isolated or in situ generated product.

As used herein, the term “pillaring” refers generally to a process that introduces stable metal oxide structures (“so-called “pillars”) between substantially parallel crystalline silicate layers. The metal oxide structures keep the silicate layers separated, creating by interlayer spacings of molecular dimensions. The term is generally used in the context of clay chemistry and are well understood by those skilled in the art of clays and zeolites, especially as applied to catalysts. In addition to the silica pillars described herein, pillared clays (also known as PILCs) with pillars comprising alumina, boron oxide, gallium oxide, tin oxide, and transition metal oxides such as chromium oxide, iron oxide, nickel oxide, titania, vanadia, zinc oxide, and/or zirconium oxide, and mixed oxides (e.g., Cr/Al, Fe/Al, Ga/Al, Si/Al. Zr/Al) are all well-known in the context of clay chemistry, as are their methods of making. Indeed, the replacement of polyaluminate cations from clays such as montmorillonite with other polycationic metal oxide materials, provides an analogous mechanism and methods for the replacement of the incorporation of polycationic precursors into the instant compositions. For illustrative examples of such pillaring reactions, structures, and catalytic activity, see, e.g., S. Cheng, “From layer compounds to catalytic materials,” Catalysis Today, 49 (1999) 303-312; E. A. Eman, “Clays as Catalysts in Petroleum Refining Industry,” ARPN J. of Sci. and Tech., 4 (4) 2013, pp 356-375; R. T. Yang, et al., “Pillared Clays as Superior Catalysts for Selective Catalytic Reduction of Nitric Oxide,” Final Technical Report, DE-FG22-96PC96206 (2000); J. T. Kloprogge, et al., “A review of the synthesis and characterisation of pillared clays and related porous materials for cracking of vegetable oils to produce biofuels,” Environmental Geology, 2005; M. Kurian, et al., “A Review of the Importance of Pillared Interlayered Clays in Green Chemical Catalysis,” IOSR J. Applied Chem., (2016); M. Kurian, “Catalysis by Pillared Montmorillonites Exchanged with Transition Metals,” Doctoral Thesis, Chochin University, 2004; G. Mata, et al., “Chromium-spaonite clay catalysts: Preparation, characterization, and catalytic performance in propene oxidation,” Applied Catalysis A: General 327 (2007) 1-12; C. B. Molina, et al., “A comparison of Al—Fe and Zr—Fe pillared clays for catalytic wet peroxide oxidation,” Chemical Engineering Journal 118 (2006) 29-35; I. Palinko, et al., “Mixed-metal pillared layer clays and their pillaring precursors,” J. Chem. Soc., Faraday Trans., 1997, 93(8), 1591-1599; S. Perathoner, et al., “Catalysts based on pillared interlayered clays for the selective catalytic reduction of NO,” Clay Minerals (1997) 32, 123-134; Purabai Kar, “Preparation, Characterization and Catalytic Applications of Pillared Clay Analogues and Clay-Polymer Composite Materials,” Doctoral Thesis, National Institute of Technology, Rourkela (2014). Each of these references is incorporated by reference, at least for their teachings of methods of analogous PILC structures, and methods of making and using the same in catalytic and separation applications.

Pillared zeolites are also known, and given the analogy of the present structures to both pillared clays and zeolites, the compositions of the present disclosure are reasonably expected. See, e.g., Trees De Baerdemaeker, et al., “A new class of solid Lewis acid catalysts based on interlayer expansion of layered silicates of the RUB-36 type with heteroatoms,” J. Mater. Chem. A, 2014, 2, 9709-9717, which describes the use of an Fe salt instead of a silylating agent was used to incorporate iron oxide fill up the linking sites in between the layers. Such structures are known to be useful as absorbents/adsorbents for gases and liquids, a variety of catalytic transformations, hydrocarbon cracking and reforming, vegetable oil cracking to form biofuels, oxidation of VOCs, methanol conversion to hydrocarbons, wet (peroxide) oxidation of phenols, and catalytic reductions of NOx. See, e.g., A. Gil, et al., Eds., Pillared Clays and Related Catalysts, Springer, 2010 (isbn=1441966706), and other references cited herein.

The terms “separating” or “separated” carries their ordinary meaning as would be understood by the skilled artisan, insofar as it connotes separating or isolating the product material from other starting materials or co-products or side-products (impurities) associated with the reaction conditions yielding the material. As such, it infers that the skilled artisan at least recognizes the existence of the product and takes specific action to separate or isolate it. Absolute purity is not required, though preferred, as the material may contain minor amounts of impurities and the separated or isolated material may contain residual solvent or be dissolved within a solvent used in the reaction or subsequent purification of the material.

As used herein, the term “crystalline microporous solids” or “crystalline microporous silicate solids,” are crystalline structures having very regular pore structures of molecular dimensions, i.e., under 2 nm. The term “molecular sieve” refers to the ability of the material to selectively sort molecules based primarily on a size exclusion process. The maximum size of the species that can enter the pores of a crystalline microporous solid is controlled by the dimensions of the channels. These are conventionally defined by the ring size of the aperture, where, for example, the term “8-MR” or “8-membered ring” refers to a closed loop that is typically built from eight tetrahedrally coordinated silicon (or aluminum) atoms and 8 oxygen atoms. In the present case, the structures described comprise 8- or 8- and 10-membered rings (designated 8-MR, 8-/10-MR, respectively). These rings are not necessarily symmetrical, due to a variety of effects including strain induced by the bonding between units that are needed to produce the overall structure, or coordination of some of the oxygen atoms of the rings to cations within the structure. The term “silicate” refers to any composition including silica. It is a general term encompassing, for example, pure-silicates, aluminosilicates, stannosilicates, etc.

The following listing of embodiments is intended to complement, rather than displace or supersede, the previous descriptions.

### Embodiment 1

A crystalline microporous silicate, designated CIT-10, which exhibits a powder X-ray diffraction (XRD) pattern exhibiting at least five of the characteristic peaks at 7.6±0.2°, 8.7±0.2°, 10.3±0.2°, 18.8±0.2°, 20.3±0.2°, 21.8±0.2°, 22.4±0.2°, 22.7±0.2°, 22.9±0.2°, and 23.6±0.2° 2-theta. In some independent Aspects of this Embodiment, the crystalline microporous silicate exhibits a powder X-ray diffraction (XRD) pattern exhibiting at least 5, 6, 7, 8, 9, or 10 of these characteristic peaks. In other independent Aspects of this Embodiment, the crystalline microporous silicate exhibits a powder X-ray diffraction (XRD) pattern exhibiting at least 5, 6, 7, 8, 9, or 10 of the characteristic peaks listed in Table 2. In some Aspects of this Embodiment, the crystalline microporous silicate exhibits a powder X-ray diffraction (XRD) pattern substantially the same as that shown in FIGS. 1 and 9A, allowing for variances in the relative intensities of the peaks.

### Embodiment 2

The crystalline microporous silicate of Embodiment 1, wherein the crystalline microporous silicate comprises a two dimensional layered structure, having an organic material, preferably a cationic organic material including any one of the OSDAs described in this context, positioned between individual crystalline silicate layers. In some Aspects of this Embodiment, the crystalline microporous silicate can be treated to act as a precursor for the topotactic condensation to a crystalline microporous silicate of RTH topology or for the pillaring using silylating and other reagents as otherwise described herein.

### Embodiment 3

The crystalline microporous silicate of Embodiment 1 or 2, having a structure which is ordered in its a and b directions (i.e., along its two dimensional crystalline layer), but which exhibits some disorder in the c direction (i.e., between layers) as evidenced by RED (rotating electron diffraction) structure analysis. In some Aspects of this Embodiment, the crystalline microporous silicate RED structure analysis such as shown in FIG. 6.

### Embodiment 4

The crystalline microporous silicate of any one of Embodiments 1 to 3, which exhibits an ²⁹Si-MAS NMR spectrum having chemical shifts of −113 ppm, −107 ppm, and −102 ppm, relative to tetramethylsilane (TMS).

### Embodiment 5

The crystalline microporous silicate of Embodiment 4 in which the chemical shifts of −113 ppm, −107 ppm, and −102 ppm have relative approximate area ratios of 8:5:3. In other Aspects of this Embodiment, the crystalline microporous silicate exhibits an ²⁹Si-MAS NMR spectrum substantially similar to that shown in FIGS. 4(A-B).

### Embodiment 6

The crystalline microporous silicate of any one of Embodiments 1 to 5, comprising an occluded or interlayered organic structure directing agent (OSDA). In some Aspects of this Embodiment, the OSDA comprises a diquaternary (dicationic) structure of:

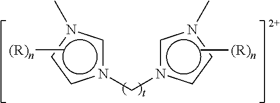

wherein t is 3, 4, 5, or 6, preferably 4 or 5; and

R is independently methyl or ethyl, preferably methyl or mainly methyl, and n is independently 1, 2, or 3; said linked pair of quaternary imidazolium cations having associated fluoride or hydroxide ions, preferably substantially free of other halide counterions, i.e., bromide, chloride, or iodide. In specific Aspects of this Embodiment, the OSDA comprises a compound having a structure:

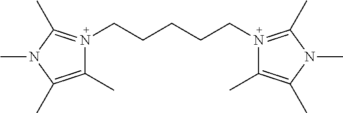

As used herein, the term “linked pair of quaternary imidazolium cations” is intended to connote that two quaternary imidazolium cations are linked by the carbon linker, and not that the two quaternized cations are necessarily identical, though this is preferred.

### Embodiment 7

The crystalline microporous silicate of any one of Embodiments 1 to 6, further optionally comprising fluoride ion. In some independent Aspects of this Embodiment, the crystalline microporous silicate comprises fluoride ion. In other independent Aspects, the crystalline microporous silicate is substantially free of fluoride ion.

### Embodiment 8

A crystalline microporous silicate, designated CIT-11, comprising a pre-calcined pillared structure (which in some cases may comprise polyoxides(hydroxides) of the pillaring metals or metalloids described elsewherein herein), in which the pillars separate substantially parallel crystalline silicate layers. In some embodiments, the crystalline microporous silicate exhibits a powder X-ray diffraction (XRD) pattern exhibiting at least five of the characteristic peaks at 6.9±0.2°, 8.6±0.2°, 10.2, ±0.2°, 15.8±0.2°, 17.3±0.2°, 18.9±0.2°, 20.3±0.2°, 21.0±0.2°, 22.2±0.2°, 25.6±0.2°, and 30.8±0.2° 2-theta. In some independent Aspects of this Embodiment, the crystalline microporous silicate exhibits a powder X-ray diffraction (XRD) pattern exhibiting at least 5, 6, 7, 8, 9, or 10 of these characteristic peaks. In other independent Aspects of this Embodiment, the crystalline microporous silicate exhibits a powder X-ray diffraction (XRD) pattern exhibiting at least 5, 6, 7, 8, 9, or 10 of the characteristic peaks listed in Table 2. In some Aspects of this Embodiment, the crystalline microporous silicate exhibits a powder X-ray diffraction (XRD) pattern substantially the same as that shown in FIG. 9B, allowing for variances in the relative intensities of the peaks. In some Aspects of this Embodiment, the pre-calcined pillars comprise precursors to oxides of aluminum, boron, gallium, silicon, tin, or transition metals such as chromium, iron, nickel, titanium, vanadium, zinc, and/or zirconium, or a mixed oxide of the elements Cr/Al, Fe/Al, Ga/Al, Si/Al, or Zr/Al.

### Embodiment 9

The crystalline microporous silicate of Embodiment 8, which exhibits an ²⁹Si-MAS NMR spectrum having chemical shifts of −113.5 ppm, −108.4 ppm, −104.5 ppm, and −15.3 ppm, relative to tetramethylsilane (TMS).

### Embodiment 10

The crystalline microporous silicate of Embodiment 9, in which the chemical shifts of −113.5 ppm, −108.4 ppm, −104.5 ppm, and −15.3 ppm have relative approximate area ratios of approximately 20:8:2:5. In other Aspects of this Embodiment, the crystalline microporous silicate exhibits a ²⁹Si-MAS NMR spectrum substantially similar to that shown in FIG. 4D.

### Embodiment 11

The crystalline microporous silicate of any one of Embodiments 8 to 10, prepared by reacting the crystalline microporous silicate of any one of Embodiments 1 to 7, with a silylating agent in the presence of an acid and an alcohol. In certain Aspects of this Embodiment, the crystalline microporous silicate of any one of Embodiments 1 to 7 may be reacted with an oxide precursor to produce a pillared structure, wherein the pillars comprise oxides of aluminum, boron, gallium, silicon, tin, or transition metals such as chromium, iron, nickel, titanium, vanadium, zinc, and/or zirconium, or a mixed oxide of the elements Cr/Al, Fe/Al, Ga/Al, Si/Al, or Zr/Al. Such oxide precursors are well known in the art of zeolite and PILC, and may include alkoxides, oxides, hydroxides, or salts (e.g., halides or carboxylates) of the corresponding metals.

### Embodiment 12

A crystalline microporous silicate, designated CIT-12, which is a calcined pillared structure of parallel silicate layers. In some embodiments, the crystalline microporous silicate exhibits a powder X-ray diffraction (XRD) pattern exhibiting at least five of the characteristic peaks at 7.7±0.2°, 8.8±0.2°, 10.3±0.2°, 18.1±0.2°, 19.3±0.2°, 20.7±0.2°, 22.6±0.2°, 25.6±0.2°, 28.5±0.2°, and 31.1±0.2° 2-theta. In some independent Aspects of this Embodiment, the crystalline microporous silicate exhibits a powder X-ray diffraction (XRD) pattern exhibiting at least 5, 6, 7, 8, 9, or 10 of these characteristic peaks. In other independent Aspects of this Embodiment, the crystalline microporous silicate exhibits a powder X-ray diffraction (XRD) pattern exhibiting at least 5, 6, 7, 8, 9, or 10 of the characteristic peaks listed in Table 2. In some Aspects of this Embodiment, the crystalline microporous silicate exhibits a powder X-ray diffraction (XRD) pattern substantially the same as that shown in FIG. 9C, allowing for variances in the relative intensities of the peaks. In some Aspects of this Embodiment, the calcined pillars comprise oxides of aluminum, boron, gallium, silicon oxide, tin oxide, or transition metal such as chromium, iron, nickel, titanium, vanadium, zinc, and/or zirconium, or a mixed oxide of the elements Cr/Al, Fe/Al, Ga/Al, Si/Al, or Zr/Al.

### Embodiment 13

The crystalline microporous silicate of Embodiment 12, which exhibits a broad resonance in an ²⁹Si-MAS NMR spectrum at chemical shifts of about −110 ppm relative to tetramethylsilane (TMS). In certain Aspects of this Embodiment, the crystalline microporous silicate exhibits an ²⁹Si-MAS NMR spectrum substantially similar to that shown in FIG. 4E.

### Embodiment 14

A process comprising heating the crystalline microporous silicate of any one of Embodiments 1 to 7 (CIT-10) to at least one temperature in a range of from 300° C. to 800° C. for a time sufficient to provide a crystalline microporous silicate of RTH topology. In certain independent Aspects of this Embodiment, the at least one temperature exceeds 380° C., 430° C., 480° C., 530° C., or 580° C. In other Aspects, the upper end of the temperature range is 800° C., 750° C., 700° C., 650° C., or 600° C.

### Embodiment 15

A process (known as pillaring) comprising reacting the crystalline microporous silicate of any one of Embodiments 1 to 6 (CIT-10) with a silylating agent (or other elemental oxide precursor) under conditions sufficient to produce the crystalline material of any one of Embodiments 7 to 9 (CIT-11). In certain Aspects of this Embodiment, the silylating agent comprises those known to be useful for pillaring such structures, for example including dichlorodimethylsilane and/or diethoxydimethylsilane]. In certain other Aspects of this Embodiment, other metal oxide precursors may also be used in this capacity, for example, a source of aluminum oxide, boron oxide, chromium oxide, iron oxide, titania, tin oxide, zinc oxide, and/or zirconium oxide in the presence of a strong acid, such as nitric or hydrochloric acid. See, e.g., Trees De Baerdemaeker, et al., “A new class of solid Lewis acid catalysts based on interlayer expansion of layered silicates of the RUB-36 type with heteroatoms,” J. Mater. Chem. A, 2014, 2, 9709-9717, which describes the use of an Fe salt instead of a silylating agent was used to incorporate iron oxide fill up the linking sites in between the layers.

### Embodiment 16

A process comprising heating the crystalline microporous silicate of any one of Embodiments 8 to 10 (CIT-11) to at least one temperature in a range of from 300° C. to 800° C. for a time sufficient to provide a crystalline microporous silicate of Embodiment 12 or 13 (CIT-12). In certain independent Aspects of this Embodiment, the at least one temperature exceeds 380° C., 430° C., 480° C., 530° C., or 580° C. In other Aspects, the upper end of the temperature range is 800° C., 750° C., 700° C., 650° C., or 600° C.

### Embodiment 17

The process of Embodiment 16, further comprising:

(a) treating the crystalline microporous silicate of Embodiment 12 or 13 (CIT-12) with an aqueous alkali, alkaline earth, transition metal, rare earth metal, ammonium or alkylammonium salt, as described elsewhere herein; and/or

(b) treating the crystalline microporous silicate of Embodiment 12 or 13 (CIT-12) with at least one type of transition metal or transition metal oxide, as described elsewhere herein.

### Embodiment 18

A process for affecting an organic transformation, the process comprising:

(a) carbonylating DME with CO at low temperatures;

(b) reducing NOx with methane or an olefin in the presence of oxygen:

(c) cracking, hydrocracking, or dehydrogenating a hydrocarbon;

(d) dewaxing a hydrocarbon feedstock;

(d) converting paraffins to aromatics:

(e) isomerizing or disproportionating an aromatic feedstock;

(f) alkylating an aromatic hydrocarbon;

(g) oligomerizing an alkene;

(h) aminating a lower alcohol;

(i) separating and sorbing a lower alkane from a hydrocarbon feedstock;

(j) isomerizing an olefin;

(k) producing a higher molecular weight hydrocarbon from lower molecular weight hydrocarbon;

(l) reforming a hydrocarbon

(m) converting a lower alcohol or other oxygenated hydrocarbon to produce an olefin products (including MTO);

(n) epoxiding olefins with hydrogen peroxide;

(o) reducing the content of an oxide of nitrogen contained in a gas stream in the presence of oxygen;

(p) converting synthesis gas containing hydrogen and carbon monoxide to a hydrocarbon stream;

(q) reducing the concentration of an organic halide in an initial hydrocarbon product;

(r) wet (peroxide) oxidation of phenols; or

(s) cracking of vegetable oils to produce biofuels;

by contacting the respective feedstock with the a catalyst comprising the crystalline microporous silicate composition of any one of Embodiments 12 to 14, under conditions sufficient to affect the named transformation.

### Embodiment 19

The process of embodiment 18 comprising reducing NOx with methane or an olefin in the presence of oxygen.

### Embodiment 20

The process of Embodiment 17 comprising converting a lower alcohol or other oxygenated hydrocarbon to an olefin product.

### Embodiment 21

A process for removing of H₂O, CO₂ and SO₂ from fluid streams, such as low-grade natural gas streams, and separating gases, including noble gases, N₂, 02, fluorochemicals formaldehyde, and lower alkanes, from gas streams, the process comprising contacting the fluid or gas stream with a composition comprising the crystalline microporous silicate composition of any one of Embodiments 12 to 14.

### EXAMPLES

The following Examples are provided to illustrate some of the concepts described within this disclosure. While each Example is considered to provide specific individual embodiments of composition, methods of preparation and use, none of the Examples should be considered to limit the more general embodiments described herein.

In the following examples, efforts have been made to ensure accuracy with respect to numbers used (e.g. amounts, temperature, etc.) but some experimental error and deviation should be accounted for. Unless indicated otherwise, temperature is in degrees Celsius, pressure is at or near atmospheric.

### Example 1

### Materials and Methods

Unless otherwise noted all reagents were purchased from Sigma-Aldrich and were used as received. Hydroxide ion exchanges were performed using Supelco Dowex Monosphere 550A UPW hydroxide exchange resin with an exchange capacity of 1.1 meq/mL. Titrations were performed using a Mettler-Toledo DL22 autotitrator using 0.01 M HCl as the titrant. All liquid NMR spectra were recorded with a 400 MHz Varian Spectrometer.

### Example 2

### OSDA Synthesis

The diquaternary OSDA used in this work:

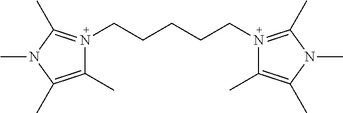

was synthesized by reacting 200 mmol of 1,2,4,5-tetramethylimidazole (TCI Chemicals) with 100 mmol of 1,5-dibromopentane (Aldrich) at reflux in methanol overnight. The solvent was then removed using rotary evaporation and the product washed with ether. The product was verified using ¹³C NMR in D₂O with methanol added as an internal standard. ¹³C-NMR (125 MHz, D₂O): δ 7.76, 7.82, 9.61, 22.82, 28.58, 31.42, 44.72, 124.84, 126.03, 141.95. The product was ion exchanged to hydroxide form using Dowex Marathon A exchange resin and the final product concentration was determined using a Mettler-Toledo DL22 autotitrator using 0.01 M HCl as the titrant.

### Example 3

### Synthesis of Crystalline Microporous Silicates

### Example 3.1

### Synthesis of CIT-10

Tetraethylorthosilicate was added to the OSDA in its hydroxide form in a Teflon Parr Reactor. The container was closed and stirred overnight to allow for complete hydrolysis. The lid was then removed, and the ethanol and some water were allowed to evaporate under a stream of air. Once the gel was dry, a small amount of water was added to obtain a homogenous liquid. Then aqueous HF was added and the mixture was stirred by hand. A second evaporation step was then used to give a final gel molar ratio of 1SiO₂:0.5R_{1/2}(OH):0.5HF:4H₂O. Seeds of CIT-10 were then added and the autoclave was sealed and placed in a rotating oven (43 rpm) at 175° C. Aliquots of the material were taken periodically by first quenching the reactor in water and then removing enough material for powder X-ray diffraction (PXRD). Synthesis times for pure silica RTH were on the order of 20 days when no seeds were added and 10 days when seeds were added. The product was recovered via centrifugation and was washed with water 3 times, a final time with acetone and dried in air at 100° C.

### Example 3.2

### Synthesis of Pure-Silica RTH

The as-made CIT-10 material prepared in Example 3.1 was calcined in breathing grade air. The material was heated to 150° C. at 1° C./min, held for three hours, then heated to 580° C. at 1° C./min and held for six hours to assure complete combustion of the organic.

### Example 3.3

### Pillaring of CIT-10 to Produce CIT-11

The procedure that led to the pillared product with the highest crystallinity (judged using PXRD) was found to be as follows. A 500 mg sample of CIT-10 was placed in a 45 mL Teflon Parr Reactor, to which 20 g of a 1.25 M solution of HCl in ethanol was added. Finally 500 mg of silyating agent (dichlorodimethylsilane or diethoxydimethylsilane, both were found to produce a similar product) was added. The reactor was sealed and place in a rotating oven at 175° C. for 24 hours. The product was recovered via centrifugation and was washed one time with absolute ethanol, three times with water and finally one time with acetone and then dried in air at 100° C.

### Example 3.3

### Synthesis of CIT-12

CIT-12 was obtained by the calcination of CIT-11 using the procedure described in Example 3.2. The as-made CIT-11 material prepared in Example 3.3 was calcined in breathing grade air. The material was heated to 150° C. at 1° C./min, held for three hours, then heated to 580° C. at 1° C./min and held for six hours to assure complete combustion of the organic.

### Example 4

### Characterizations

Liquid NMR spectra were recorded with a 500 MHz spectrometer. ¹³C and ²⁹Si solid-state NMR were performed using a Bruker DSX-500 spectrometer (11.7 T) and a Bruker 4 mm MAS probe. The spectral operating frequencies were 500.2 MHz, 125.721 MHz and 99.325 MHz for ¹H, ¹³C and ²⁹Si nuclei, respectively. Spectra were referenced to external standards as follows: tetramethylsilane (TMS) for ¹H and ²⁹Si and adamantane for ¹³C as a secondary external standard relative to tetramethylsilane. Samples were spun at 8 kHz for ¹³C and ²⁹Si MAS and CPMAS NMR experiments. Chemical shift variances are estimated to be less than 0.2 ppm. Thermogravimetric analysis measurements were performed on Netzsch STA 449C Jupiter. Samples were heated in air to 900° C. at a rate of 1° C./min. Argon physical adsorption isotherms were performed at 87 K using a Quantachrome Autosorb iQ and were conducted using a quasi-equilibrium, volumetric technique. PXRD data were collected on a Rigaku MiniFlex II with Cu Kα radiation. Variable temperature PXRD patterns were collected from 30° C. to 580° C. at increments of 50° C. under ambient conditions, using a PANalytical Empyrean powder diffractometer (Cu Kα radiation) equipped with an Anton Paar HTK 1200N high-temperature chamber. The sample was stabilized at each measurement temperature for 15 min before starting each measurement. The temperature ramp between two consecutive temperatures was 5° C./min. SEM images were acquired on a ZEISS 1550 VP FESEM, equipped with in-lens SE. EDS spectra were acquired with an Oxford X-Max SDD ray Energy Dispersive Spectrometer system. Three-dimensional electron diffraction data were collected using the rotation electron diffraction (RED) technique. The RED software was installed on a JEOL 2010 microscope operating at 200 kV, and data were collected over a tilt range of ±50° with a tilt step of 0.50°, the exposure time is 3 seconds per tilt step.

### Example 5

### Results and Discussions

### Example 5.1

### Synthesis of CIT-10 and Calcination to Produce Pure-Silica RTH

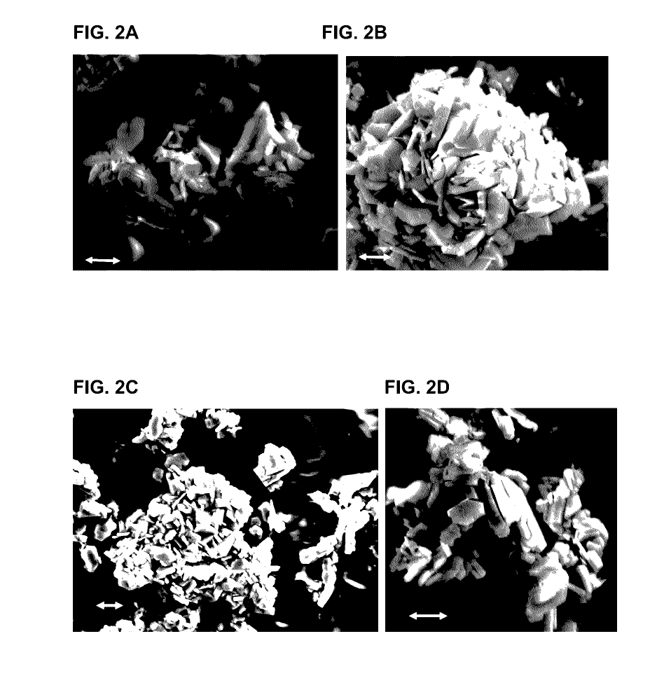

Imidazolium OSDAs in the synthesis of microporous materials have been found to be useful in producing a wide range of crystalline phases including LTA, RTH, STW, CSV and HEU in addition to a number of additional phases discussed in the previous references. While the majority of these products are microporous materials that were made with OSDAs intact inside the framework, the high-silica HEU (CIT-8) could be prepared from a layered precursor (CIT-8P). CIT-8P was synthesized in fluoride-media from a gel containing a relatively high amount of aluminum (gel Si/Al=15 or 20). The result of finding a layered material in these conditions led the inventors to continue to explore similar inorganic conditions. In aluminum-free syntheses, the present inventors have reported that diquats formed from tetramethylimidazole can be used to prepare pure-silica CSV (CIT-7). See, e.g., U.S. patent application Ser. No. 14/602,415, filed Jan. 22, 2015. However, under similar conditions to that used in aluminosilicate systems, in pure silicate systems, the diquat containing a five-carbon chain linker length was found to lead to a phase that could not be identified (shown in FIG. 1). Upon calcination, this material yielded a phase that was easily identified as pure-silica RTH (FIG. 1). This is the second reported method to synthesize pure-silica RTH, and may broaden its use as previously synthesis of this type of material required the use of a difficult to prepare OSDA. SEM images of CIT-10 and pure-silica RTH are shown in FIG. 2.

These images did not show a regular morphology that is commonly observed in highly crystalline materials, but instead showed morphology resembling thick plates. Plate-like morphology is common in layered materials, but the thickness of the plates in these samples was unusual.

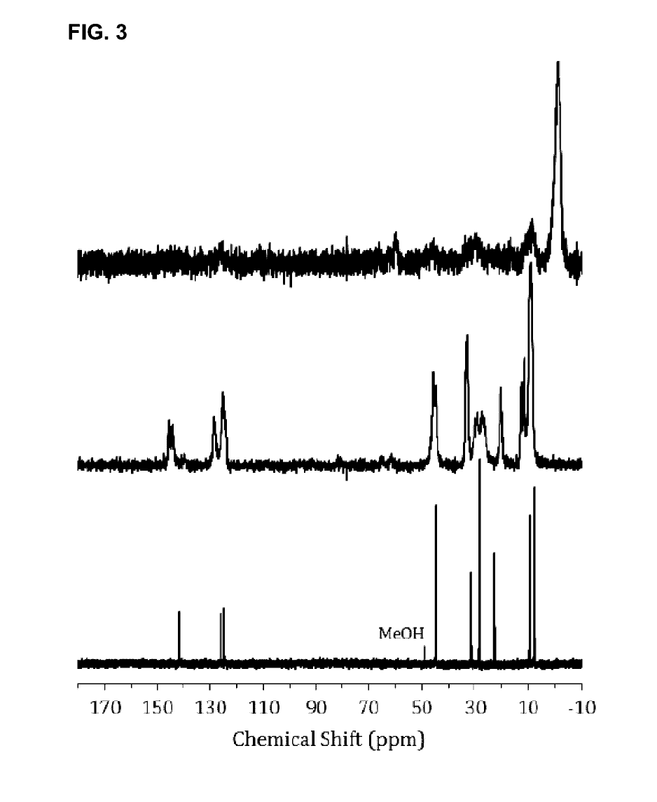

To determine the mechanism of formation of pure-silica RTH, the materials were studied using ¹³C CPMAS NMR, ²⁹Si MAS and CPMAS NMR and variable temperature PXRD. The ¹³C CPMAS NMR of CIT-10 (FIG. 3) showed that the diquat OSDA was occluded intact in the material. Notably, many of the peaks in this spectrum were split, indicating that otherwise equivalent carbon atoms were present in non-equivalent environments; this has been previously reported in layered materials.

The ²⁹Si MAS and CPMAS NMRs of CIT-10 are shown in FIG. 4. CIT-10 was also studied using CPMAS NMR in addition to MAS NMR to confirm the resonances (organic-containing materials often exhibit a poor signal-to-noise ratio). In the as-made material there are three resonances at −113, −107 and −102 ppm with approximate area ratios of 8:5:3. The signals at −113 and −107 ppm were assigned to Q4 silicon, Si(OSi)₄ coordination, while the signal at −102 ppm was assigned to Q3 silicon, Si(OSi)3(OH) coordination. The presence of Q3 silicon is expected in a layered material. The ratio of Q3/Q4 silicon in the as made material is 0.23, which is very close to the theoretical value of 0.25. Upon calcination, the ²⁹Si MAS NMR no longer showed the presence of any Q3 silicon and instead showed 3 resonances in the Q4 region at −116, −114 and −109 ppm, with area ratios of 1:2:1. These area ratios agreed with the crystal structure of RTH, as it contains 4 independent T-sites.

The structural mechanism of condensation was determined by using variable temperature PXRD as well as RED. The variable temperature PXRD of CIT-10 is shown in FIG. 5. When compared with the PXRD patterns of RTH in FIG. 1 (labelled with the crystallographic indices), it is apparent that peak positions for hk0 reflections remain during heating, while the peak positions for the hk1 (1#)) reflections are shifted to higher 20 angles (i.e., lower d-spacing). This result indicated that the 3D RTH structure formed via topotactic condensation along the c-axis, and that the a and b axes were intact in the layered material. The structural change was further confirmed by studying CIT-10 using RED (FIG. 6). The RED clearly showed that CIT-10 was ordered in the a and b directions (indicated by clearly defined diffraction spots), but that some disorder is present in the c direction (indicated by diffraction streaks between diffraction spots). Thus, results from using both techniques confirm that CIT-10 contains 2D sheets in the a and b directions that are separated by a disordered organic in the c direction.

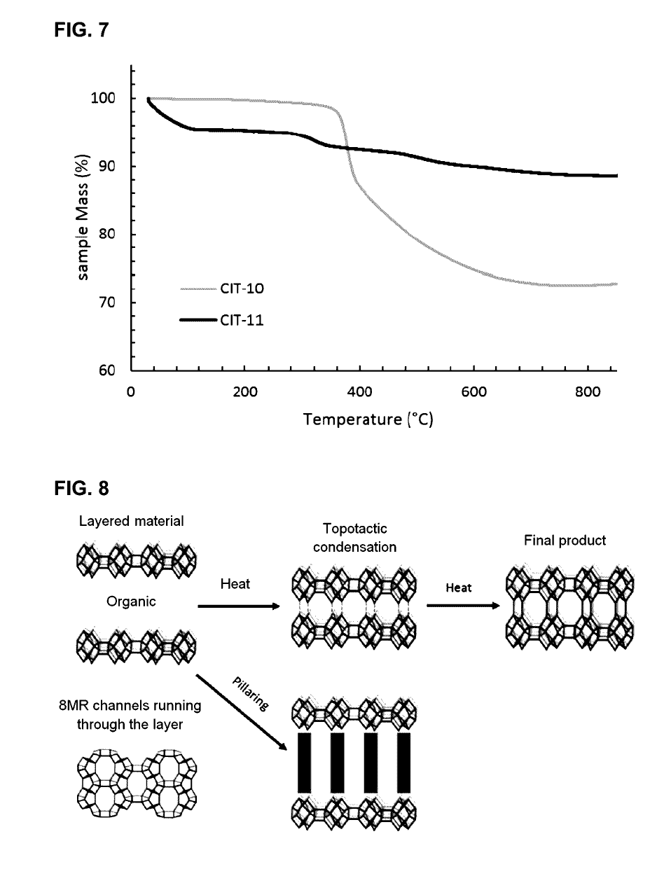

The TGA data complemented the condensation temperature observed in the variable temperature PXRD. In the variable temperature PXRD, the structure of the layered material was intact until 330° C., then the low angle peak corresponding to the 001 direction abruptly disappeared. This reflection was absent at the PXRD pattern taken at 380° C., then began to emerge around 430° C. From the TGA trace in FIG. 7, a sharp mass loss occurring around 375° C. was seen, and was in the same temperature range where the low angle peak disappeared in the variable temperature PXRD. The rapid change observed with RTH is in contrast to the TGA trace and structural changes observed with CIT-8P where a gradual shift in position of the low-angle peak was observed along with the gradual decrease in mass.

CIT-10 has an 8MR channel running through the layer along the c axis, with dimensions of 2.5×5.6 Å. As the structure condensed along the c-axis, a second 8MR channel system running through the a-axis was formed, and a cavity was created at the intersection of the two 8MR channels, forming the RTH framework structure. The condensation process is shown schematically in FIG. 8. As the schemes in the figure depict, the RTH layer was actually a half unit cell thick compared to the final RTH framework unit cell.

Table 1 shows the comparisons of the d-spacing corresponding to the first and most intense PXRD peak for known 2D layered materials, and the corresponding d-spacing shrinkage after topotactic condensation to form 3D framework materials. In most of the cases (including CIT-10) the d-spacing shrinkage due to topotactic condensation was around 2 Å, an observation that has been discussed by others. It is also interesting to note that although the OSDAs used to make CIT-10 and CIT-8P were very similar, the latter demonstrated a d-spacing contraction nearly twice that of the former.

TABLE 1
Comparisons of the d-spacing corresponding to the first and most intense
PXRD peak for known 2D layered materials, and the corresponding d-spacing
shrinkage after topotactic condensation to form 3D framework materials.

| | | | Corresponding | d-spacing | |
|---|---|---|---|---|---|
| 2D Zeolite | d-spacing (Å) | 3D Zeolite | d-spacing (Å) | Shrinkage (Å) | Ref.* |
| CIT-10 | 11.8 | Siliceous RTH | 9.8 | 2.0 | This work |
| RUB-36 | 11.1 | RUB-37 (CDO) | 9.2 | 1.9 | [39] |
| MCM-22P | 26.9 | MCM-22 | 24.9 | 2.0 | [17, 45] |
| | | (MWW) | | | |
| HMP-2 | 17.5 | MCM-35 (MTF) | 15.4 | 2.1 | [22] |
| RUB-39 | 10.8 | RUB-41 (RRO) | 8.7 | 2.1 | [25, 39] |
| R-RUB-18 | 9.1 | RUB-24 (RWR) | 6.8 | 2.3 | [26] |
| EU-19 | 11.5 | EU-20 (CAS- | 8.3 | 3.2 | [20] |
| | | NSI) | | | |
| PREFER | 13.1 | FER | 9.4 | 3.7 | [16] |
| CIT-8 | 12.8 | CIT-8 (HEU) | 8.9 | 3.9 | [30] |

*[16] L. Schreyek., et al., Microporous Mater., 1996, 6, 259-271; [17] M. E. Leonowicz, et al., Science, 1994, 264, 1910-1913; [20] B. Marler, et al., Microporous Mesoporous Mater., 2006, 90, 87-101; [22] T. ikeda, et al., Angew. Chem. Int. Ed. Engl., 2004, 43, 4892-4896; [25] Y. X. Wang, et al., Chem. Mater., 2005, 17, 43-49; [26] B. Marler, et al., Microporous Mesoporous Mater., 2005, 83, 201-211; [30] U. Diaz, et al., Dalton Trans., 2014, 43, 10292-10316; [39] W. Wan, et al., J. Appl. Crystallogr., 2013, 46, 1863-1873; [45] W. J. Roth, et al., Microporous Mesoporous Mater., 2011, 142, 168-177.

### Example 5.2

### Pillaring of CIT-10

In some cases it is possible to pillar layered materials using a monomeric silane in order to prepare materials with pores larger than would have been formed by topotactic condensation. These materials are commonly referred to as interlayer expanded zeolites (IEZ), and they have been prepared from precursors such as PREFER, MWW(P), PLS-1, MCM-47, RUB-36, RUB-39 and Nu-6(1). Pillaring is normally carried out in acidic media, under hydrothermal conditions, and two of the most common pillaring agents are dichlorodimethylsilane and diethoxydimethylsilane. In attempting to pillar CIT-10, a wide range of conditions were explored including acid type, aqueous versus ethanolic acid, silane source, and reaction temperature and time. The optimal conditions to pillar CIT-10 were found to be 1.25 M HCl in ethanol with either dichlorodimethylsilane or diethoxydimethylsilane at 175° C. for 24 hours. Other conditions led to what appeared to be pillared materials (based on PXRD), but these materials exhibited very weak x-ray reflections. These results suggested framework destruction, and these solids were often not stable to calcination. This phenomenon has been observed before, i.e., that acidic ethanol was the only effective medium to carry out pillaring. It has been postulated that the reason for this is that effective pillaring takes place when the rate of removal of OSDA is well matched by the rate of silylation. It should be noted that while no special efforts were made here to preclude trace amounts of water in these syntheses (such as working in a glovebox or using a Schlenk line), the water content was likely very low. The X-ray diffraction results of pillared CIT-10 are shown in FIG. 9. As can be observed from the PXRD patterns, pillaring caused a shift in the most intense reflection from 7.5° 2θ in CIT-10 to 6.8° 2θ in CIT-11 (i.e., 1.1 Å expansion). This peak continues to shift to 7.7° 2θ in CIT-12 (after calcination). The ¹³C CPMAS NMR of CIT-11 (FIG. 3) showed that the majority of the organic was removed under acidic conditions (while CPMAS NMR was not quantitative this was confirmed by TGA, shown in FIG. 7). This result was expected as a change in color of the acidic medium was observed. The strong resonance observed near −1 ppm is consistent with (CH₃)₂Si carbon that is expected from the pillaring. (Prior to NMR analysis it was necessary to degas the material under vacuum at 150° C. to remove any residual ethanol or acetone.) The TGA analysis of CIT-11 (FIG. 7) showed several distinct mass loss regions. The first mass loss of 5% was attributed to the loss of water and possibly residual ethanol or acetone (material was dried in air at 100° C. prior to analysis but not under vacuum as was used with the NMR sample). The second sharp mass loss began around 300° C., and was attributed to removal of residual organic (present in ¹³C CPMAS NMR). There was a third, distinct region of mass loss that began around 500° C. and was attributed to combustion of the Si—CH₃ groups to form hydroxyl groups.

The ²⁹Si NMR spectra were also consistent with a pillared material (FIG. 4). In CIT-10, both Q⁴ and Q³ environments are observed, consistent with a layered material (vide supra). In the pillared material, CIT-11, resonances were observed at −113.5, −108.4, −104.5 and −15.3 ppm with approximate area ratios of 20:8:2:5. The resonances at −113.5 and −108.4 are assigned to Q⁴ silicon and the resonance at −104.5 is assigned to residual Q³ silicon. The Q³/Q⁴ ratio in the pillared material is 0.07, a significant decrease from 0.23 in CIT-10, indicating that a substantial amount of Q3 species had been consumed in linking the layers of the material. The resonance at −15.3 was assigned to bridging silanol groups bonded to two methyl groups, that is Si(CH₃)₂(OSi)₂ coordination. The ratio of (Q²+Q³)/Q⁴ was 0.25, consistent with the expected value from the RTH layer. Upon calcination the material exhibited a broad resonance around −110 ppm. A single broad resonance had been observed in other pillared, calcined materials such as PLS-4. No obvious peak for Q² silicon can be seen near −90 ppm, but it is likely obscured by the much broader resonance.

The structure of CIT-11/12 is a 3D pore system consisting of 8 and 10MRs, shown in FIG. 8. The 8MR running the in the c-direction perpendicular to the RTH layer remained intact. The pillars form two new ring sizes as the previous 8MR along the a-direction expands to a 10MR and the 6MR along the b-direction expands to an 8MR. This means that the previous 2D ring system in RTH expands be a 3D ring system in CIT-11/12.

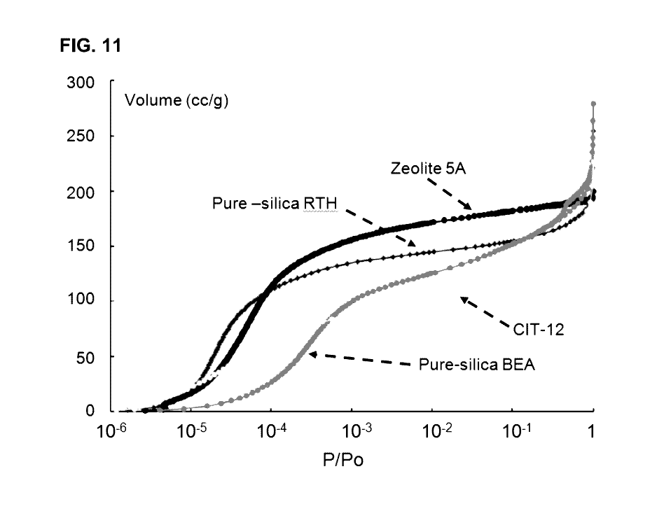

The pore system of CIT-12 was confirmed using argon adsorption. The results from this analysis are shown in FIG. 11 compared to pure-silica RTH as well as pure-silica BEA and zeolite 5A. All of these isotherms were plotted on the same graph as the shape of the isotherm in the low pressure regime is an indication of the pore size distribution. The comparison of the low pressure region of the argon adsorption isotherms indicated that this material had a pore system that has expanded compared to pure-silica RTH (8MRs) and has larger pores, closer to those of MFI (10MRs), consistent with the structure solution.

A fourth, 2D layer zeolite precursor has been synthesized, the RTH-type layer, denoted CIT-10. This is the first reported 2D layer that contains small pores that are perpendicular to the layer. Upon calcination, this material forms pure-silica RTH, making pure-silica RTH accessible without using a difficult to synthesize organic. CIT-10 can be pillared, forming CIT-11 that can then be calcined, forming CIT-12. CIT-12 contains a 3D pore system of 8 and 10MRs. CIT-10 is the first material to contain small pores running through the layer; it is possible this material could find use in separations, especially of small molecules and this layer should be hydrophobic as it is a pure-silica material. Such possibilities have already been explored with other microporous material frameworks such as LTA and MFI. However, the RTH layers are only half a unit cell thick compared to a full unit cell with MFI (which has medium pores) and multiple unit cells with LTA (small pores). Additionally the RTH layers have elliptical pores running through them, which may offer additional size discrimination compared to other small pore materials with circular pores, such as LTA.

As those skilled in the art will appreciate, numerous modifications and variations of the present invention are possible in light of these teachings, and all such are contemplated hereby. The disclosures of each patent, patent application, and publication cited or described in this document are hereby incorporated herein by reference, each in its entirety, for at least their teachings in the context in which the reference was raised.

What is claimed:

1. A crystalline microporous silicate, designated CIT-10, which exhibits a powder X-ray diffraction (XRD) pattern exhibiting at least five of the characteristic peaks at 7.6±0.2°, 8.7±0.2°, 10.3±0.2°, 18.8±0.2°, 20.3±0.2°, 21.8±0.2°, 22.4±0.2°, 22.7±0.2°, 22.9±0.2°, and 23.6±0.2° 2-theta.

2. The crystalline microporous silicate of claim 1, wherein the crystalline microporous silicate comprises a two dimensional layered structure, having an organic material sandwiched between individual crystalline silicate layers.

3. The crystalline microporous silicate of claim 2, having a structure which is ordered along its two dimensional crystalline silicate layers, but which exhibits disorder between its crystalline silicate layers, as evidenced by RED (rotating electron diffraction) structure analysis.

4. The crystalline microporous silicate of claim 1, which exhibits an ²⁹Si-MAS NMR spectrum having resonances at chemical shifts of −113 ppm, −107 ppm, and −102 ppm, relative to tetramethylsilane (TMS).

5. The crystalline microporous silicate of claim 4, wherein the resonances at chemical shifts of −113 ppm, −107 ppm, and −102 ppm have relative integrated intensities of 8, 5, and 3, respectively.

6. The crystalline microporous silicate of claim 1, comprising an occluded or interlayered organic structure directing agent (OSDA) comprising a structure of: 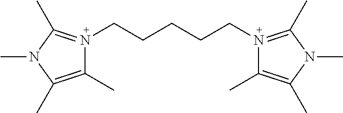
sandwiched between individual crystalline silicate layers.

7. A crystalline microporous silicate, designated CIT-11, which exhibits a powder X-ray diffraction (XRD) pattern exhibiting at least five of the characteristic peaks at 6.9±0.2°, 8.6±0.2°, 10.2, ±0.2°, 15.8±0.2°, 17.3±0.2°, 18.9±0.2°, 20.3±0.2°, 21.0±0.2°, 22.2±0.2°, 25.6±0.2°, and 30.8±0.2° 2-theta.

8. The crystalline microporous silicate of claim 7, which exhibits an ²⁹Si-MAS NMR spectrum having chemical shifts of −113.5 ppm, −108.4 ppm, −104.5 ppm, and −15.3 ppm, relative to tetramethylsilane (TMS).

9. The crystalline microporous silicate of claim 8, wherein the resonances at chemical shifts of −113.5 ppm, −108.4 ppm, −104.5 ppm, and −15.3 ppm, have relative integrated intensities of 20, 8, 2, and 5, respectively.

10. The crystalline microporous silicate of claim 7 prepared by reacting the crystalline microporous silicate of claim 1 with a silylating agent in the presence of an acid and an alcohol.

11. A crystalline microporous silicate, designated CIT-12, which exhibits a powder X-ray diffraction (XRD) pattern exhibiting at least five of the characteristic peaks at 7.7±0.2°, 8.8±0.2°, 10.3±0.2°, 18.1±0.2°, 19.3±0.2°, 20.7±0.2°, 22.6±0.2°, 25.6±0.2°, 28.5±0.2°, and 31.1±0.2° 2-theta.

12. The crystalline microporous silicate of claim 11, which exhibits a broad resonance in an ²⁹Si-MAS NMR spectrum at chemical shifts of about −110 ppm relative to tetramethylsilane (TMS).

13. A process comprising heating the crystalline microporous silicate of claim 1 to at least one temperature in a range of from 300° C. to 800° C. for a time sufficient to provide a crystalline microporous silicate of RTH topology.

14. A process comprising reacting the crystalline microporous silicate of claim 1 with a silylating agent under conditions sufficient to produce the crystalline material of claim 7.

15. The process of claim 14, wherein the silylating agent comprises dichlorodimethylsilane and/or diethoxydimethylsilane.

16. A process comprising heating the crystalline microporous silicate of claim 7 to at least one temperature in a range of from 300° C. to 800° C. for a time sufficient to provide a crystalline microporous silicate of claim 10.

17. A process for affecting an organic transformation, the process comprising:
(a) carbonylating DME with CO at low temperatures;
(b) reducing NOx with methane or an olefin in the presence of oxygen:
(c) cracking, hydrocracking, or dehydrogenating a hydrocarbon;
(d) dewaxing a hydrocarbon feedstock;
(d) converting paraffins to aromatics:
(e) isomerizing or disproportionating an aromatic feedstock;
(f) alkylating an aromatic hydrocarbon;
(g) oligomerizing an alkene;
(h) aminating a lower alcohol;
(i) separating and sorbing a lower alkane from a hydrocarbon feedstock;
(j) isomerizing an olefin;
(k) producing a higher molecular weight hydrocarbon from lower molecular weight hydrocarbon;
(l) reforming a hydrocarbon
(m) converting a lower alcohol or other oxygenated hydrocarbon to produce an olefin products (including MTO);
(n) epoxidizing olefins with hydrogen peroxide;
(o) reducing the content of an oxide of nitrogen contained in a gas stream in the presence of oxygen;
(p) converting synthesis gas containing hydrogen and carbon monoxide to a hydrocarbon stream;
(q) reducing the concentration of an organic halide in an initial hydrocarbon product;
(r) the wet (peroxide) oxidation of phenols; or
(s) cracking of vegetable oils to produce biofuels;
by contacting the respective feedstock with a catalyst comprising the crystalline microporous silicate composition of claim 11, under conditions sufficient to affect the named transformation.

18. The process of claim 17 comprising reducing NOx with methane or an olefin in the presence of oxygen.

19. The process of claim 17 comprising converting a lower alcohol or other oxygenated hydrocarbon to an olefin product.

20. A process for removing of H₂O, CO₂ and SO₂ from fluid streams, such as low-grade natural gas streams, and separating gases, including noble gases, N₂, O₂, fluorochemicals formaldehyde, and lower alkanes from gas streams, the process comprising contacting the fluid or gas stream with the crystalline microporous silicate composition of claim 11.
